(19) United States
(12) Patent Application Publication
(10) Pub. No.: US 2010/0227401 A1
(43) Pub. Date: Sep. 9, 2010

(54) MEDIA AND PROCESSES FOR THE EX VIVO PRODUCTION OF MEGAKARYOCYTES FROM HUMAN CD34+ CELLS

(75) Inventors: Chao-Ling YAO, Tainan City (TW); Te-Wei CHEN, Tainan City (TW); Shiaw-Min HWANG, Hsinchu City (TW)

Correspondence Address: THE NATH LAW GROUP, 112 South West Street, Alexandria, VA, 22314, US

(73) Assignee: FOOD INDUSTRY RESEARCH AND DEVELOPMENT INSTITUTE, Hsinchu (TW)

(21) Appl. No.: 12/754,247

(22) Filed: Apr. 5, 2010

Related U.S. Application Data
(63) Continuation-in-part of application No. 11/192,960, filed on Jul. 29, 2005, now Pat. No. 7,723,106.
Application No. 11/192,960 is a continuation-in-part of application No. 12/123,423, filed on May 19, 2008.
Application No. 12/123,423 is a division of application No. 10/909,370, filed on Aug. 3, 2004, now abandoned.
(60) Provisional application No. 60/592,042, filed on Jul. 29, 2004.
(60) Provisional application No. 60/492,741, filed on Aug. 6, 2003.

(30) Foreign Application Priority Data
Jan. 26, 2010 (TW) 099102083

(51) Int. Cl. C12N 5/071; C12N 5/02

(52) U.S. Cl. 435/372; 435/405

(57) ABSTRACT

Disclosed herein are media and processes for the ex vivo production of megakaryocytes from human CD34⁺ cells, in which human CD34⁺ cells, either being freshly isolated from a newborn's cord blood or having been subcultured in an expansion medium after isolation from a newborn's cord blood, are subjected to cultivation in a cultivating medium consisting essentially of a basal medium, a serum substitute and a cytokine cocktail, wherein the serum substitute consists of human serum albumin, insulin, and transferrin; and the cytokine cocktail consists of thrombopoietin (TPO), stem cell factor (SCF), Flt-3 ligand (FL), interleukin-3 (IL-3), IL-6, IL-9, and granulocyte-macrophage colony-stimulating factor (GM-CSF).

### CROSS-REFERENCE TO RELATED APPLICATIONS

This application claims priority of Taiwanese Application No. 099102083, filed on Jan. 26, 2010.

This application is a continuation-in-part of U.S. patent application Ser. No. 11/192,960, filed on Jul. 29, 2005, the disclosure of which is incorporated herein by reference.

U.S. patent application Ser. No. 11/192,960 claims priority from U.S. Provisional Application No. 60/592,042, filed on Jul. 29, 2004.

This application is also a continuation-in-part of U.S. patent application Ser. No. 12/123,423, filed on May 19, 2008, the disclosure of which is incorporated herein by reference.

U.S. patent application Ser. No. 12/123,423 is a divisional application of U.S. patent application Ser. No. 10/909,370, filed on Aug. 3, 2004, and abandoned.

U.S. patent application Ser. No. 10/909,370 claims priority from Provisional Application No. 60/492,741, filed on Aug. 6, 2003.

### BACKGROUND OF THE INVENTION

1. Field of the Invention

This invention relates to media and processes for the ex vivo production of megakaryocytes from human CD34⁺ cells, in which human CD34⁺ cells, either being freshly isolated from a newborn's cord blood or having been subcultured in an expansion medium after isolation from a newborn's cord blood, are subjected to cultivation in a cultivating medium consisting essentially of a basal medium, a serum substitute and a cytokine cocktail, wherein the serum substitute consists essentially of human serum albumin, insulin, and transferrin; and the cytokine cocktail consists essentially of thrombopoietin (TPO), stem cell factor (SCF), Flt-3 ligand (FL), interleukin-3 (IL-3), IL-6, IL-9, and granulocyte-macrophage colony-stimulating factor (GM-CSF).

2. Description of the Related Art

Megakaryocytes (Mks) expressing CD41a and CD61 antigens are the progenitors of platelets and play important roles with platelets in thrombosis and hemostasis. Mks are an extremely rare cell population in myeloid cells (<1%) and are generated from CD34⁺ hematopoletic stem cells (HSCs) through megakaryocytopoiesis. Mks are mainly located in bone marrow (BM), and also appear in lung, spleen, liver, cord blood (CB), and mobilized peripheral blood (MPB).

Thrombocytopenia is a disease caused by an insufficient amount of thrombocytes (also known as platelets) or Mks in the blood. This disease has often been observed in patients with hepatitis virus-related cirrhosis or after receiving high-dose chemotherapy. Patients with thrombocytopenia are at high risk for bleeding complications. To reduce the period of thrombocytopenia and accelerate platelet recovery, two treatments are commonly used. One involves the transfusion of allogeneic platelet concentrate, and the other involves the administration of thrombopoietin (TPO). However, routine transfusion of allogeneic platelet concentrate into patients puts patients at risk for infection and immunorejection, whereas the administration of TPO appears to be less effective after receiving high-dose chemotherapy.

Cord blood (CB) collected from the postpartum placenta and umbilical cord has been proven to be a rich source of HSCs and serves as an alternative to BM and MPB for hematopoietic reconstitution after chemotherapy. However, patients receiving CB transplantation require a longer period to recover platelets and neutrophils than those receiving BM or MPB transplantation. Recently, several studies have focused on the ex vivo induction and the transplantation of Mks from HSCs.

In Stem Cells, 1993, 11:120-129, Rolande Berthier et al. reported a serum-free culture medium composed of IMDM, 1.5% deionized bovine serum albumin (BSA), 300 μg/mL transferrin, 10 μg/mL insulin, 28 μg/mL calcium chloride, 2×10⁻³ M glutamine, 1×10⁻⁴ M sodium pyruvate, 10⁻⁴ M 2-mercaptoethanol (2-ME), and 40 μL/mL of a mixture of sonicated lipids. Far better growth of megakaryocyte colonies from CD34⁺ BM cells stimulated by IL-3 and IL-6 was observed in this serum-free culture medium. The optimal concentration of IL-3 alone was 5 ng/mL, and an optimal synergistic effect of IL-6 (5 ng/mL) was obtained when combined with a suboptimal dose of IL-3 (1 ng/mL).

In Blood, 1995, 86:3725-3736, R. Guerriero et al. reported that hematopoietic progenitor cells (HPCs) were induced to megakaryocytic differentiation/maturation in serum-free liquid suspension culture treated with a growth factor cocktail (IL-3, c-kit ligand, and IL-6) and/or recombinant mpl ligand (mpIL), where the serum-free liquid suspension culture was composed of IMDM supplemented with 10 mg/mL BSA, 0.7 mg/mL pure human transferrin, 40 μg/mL human low-density lipoprotein, 10 μg/mL insulin, 10⁻⁴ mol/L sodium pyruvate, 2×10⁻³ mol/L L-glutamine, rare inorganic elements supplemented with 4×10⁻⁸ mol/L iron sulphate, and nucleosides (10 μg/mL of each). The growth factor cocktail induced the growth of a 40% Mk population, i.e., 4×10⁴ cells at day 0 generated 2×10⁵ Mks at terminal maturation (day 12). Further addition of mpIL increased the Mk purity level to 80% with a final yield of 4×10⁵ Mks. Treatment with mpIL alone resulted in a 97% to 99% Mk population, with a mild increase of cell number (1.5×10⁵ cells).

In Journal of Hematotherapy, 1999, 8:199-208, Phil Lefebvre et al. reported that promegapoietin (PMP) induced megakaryocytopoietic activity comparable to that achieved with TPO plus IL-3 using CD34⁺-selected cells and might be useful for ex vivo expansion of MK for clinical trials. The culture medium was commercially available serum-free medium Easymega supplemented with 2 mM glutamine, 100 U/mL penicillin, 100 μg/mL streptomycin, and gentamicin, in which TPO was added at a concentration of 10 ng/mL, IL-3 was added at 10 ng/mL and PMP was added at 200 ng/mL.

In Chao-Ling Yao et al. (2003), Enzyme and Microbial Technology, 33:343-352, the applicants developed a serum-free, stroma-free and cytokine-containing culture system (i.e. expansion medium) for the ex vivo expansion of CD34⁺ and colony-forming cell (CFC). The experimental results show that the optimal compositions of the serum substitutes and the cytokine cocktail were BIT2 (1.5 g/L BSA, 4.39 μg/mL insulin, 60 μg/mL transferrin, and 25.94 μM 2-ME), and CC-S6 (8.46 ng/mL TPO, 4.09 ng/mL IL-3, 15 ng/mL SCF, 6.73 ng/mL FL, 0.78 ng/mL IL-6, 3.17 ng/mL G-CSF, and 1.30 ng/mL GM-CSF) in the Iscove's modified Dulbecco's medium, respectively.

The applicants further found that not only CD34⁺ cells and colony-forming cells but also CD133⁺ cells, CD34⁺CD38⁻ cells, CD34⁺CD133⁺ cells, CD34⁺CXCR4⁺ cells, CD133⁺CXCR4⁺ cells, long-term culture-initiating cells (LTC-ICs), and G₀/G₁-phase cells were highly expanded in said expansion medium (Chao-Ling Yao et al. (2006), Stem Cells and Development, 15:70-78).

In Chao-Ling Yao et al. (2004), Experimental Hematology, 32:720-727, the applicants developed a serum-free, stroma-free, and chemically defined medium for the expansion of hematopoietic stem cell (HSC). The experimental results show that the optimal compositions of serum substitutes and the cytokine cocktail for HSC expansion in the MNC culture system were BIT (4 g/L BSA, 0.71 μg/mL insulin, and 27.81 μg/mL transferrin), and CC-9 (5.53 ng/mL TPO, 2.03 ng/mL IL-3, 16 ng/mL SCF, 4.43 ng/mL FL, 2.36 ng/mL IL-6, 1.91 ng/mL G-CSF, 1.56 ng/mL GM-CSF, 2.64 ng/mL SCGF, and 0.69 ng/mL IL-11) in the Iscove's modified Dulbecco's medium.

In Haematologica, 2004, 89:630-631, Stefan Scheding et al., generated megakaryocytic cells from CliniMACS-CD34⁺-selected cells ex vivo using X-VIVO10 medium supplemented with 100 ng/mL TPO, 10 ng/mL interleukin-3 (IL-3), and 10 ng/mL stem cell factor (SCF) and investigated the feasibility of the large-scale expansion and transplantation of autologous megakaryocytic cells in four patients with advanced solid tumor.

In U.S. Patent Publication No. 20050032122, which is the laid-open publication of U.S. patent application Ser. No. 10/909,370, the applicants developed a method of determining the optimal composition of a serum-free, eukaryotic cell culture medium supplement, using 2-level factorial design and the deepest ascent method. The applicants further developed a serum-free, eukaryotic cell culture medium capable of supporting the growth of the CD34⁺ hematopoietic cells, which comprises basal medium IMDM, 10% FBS, 32.1 ng/mL TPO, 20 ng/mL IL-3, 30.5 ng/mL SCF, and 22.3 ng/mL FL.

In STEM CELLS, 2006, 24:2877-2887, Takuya Matsunaga et al. generated platelets from CB CD34⁺ cells using a three-phase culture system. Five hundred CB CD34⁺ cells were cultured on telomerase gene-transduced human stromal cells (hTERT stroma) in serum-free medium supplemented with SCF, Flt-3/Flk-2 ligand (FL) and TPO to expand hematopoietic progenitor/stem cells (first phase). The expanded cells were further cultured in the presence of 10 ng/mL SCF, 50 ng/mL FL, 50 ng/mL TPO and 20 ng/mL IL-11 on hTERT stroma to give rise to megakaryocytic lineage differentiation and expansion (second phase) and finally cultured in a liquid culture system containing SCF, FL, TPO, and IL-11 to generate platelets from megakaryocytes (third phase). With this system, Takuya Matsunaga et al. succeeded in producing an estimated 1.68×10¹¹ platelets from 5×10⁶ CD34⁺ cells.

In U.S. Patent Publication No. 20060024827, which is the laid-open publication of U.S. patent application Ser. No. 11/192,920, the applicants developed a stroma-free, serum-free, and chemically defined medium for the ex vivo expansion of mononuclear cells, in particular hematopoietic stem cells, such as CD34⁺ cells. The chemically defined medium comprises a basal medium, a serum substitute and a cytokine formula, in which the basal medium may be Iscove's modified Dulbecco's medium (IMDM), McCoy's 5A medium, minimum essential medium alpha medium (α-MEM), or F-12K nutrient mixture medium (Kaighn's modification, F-12K); the serum substitute includes bovine serum albumin (BSA), insulin, and transferrin (TF); and the cytokine formula includes thrombopoietin (TPO), stem cell factor (SCF), stem cell growth factor-α (SCGF), Flt-3 ligand (FL), IL-3, IL-6, IL-11, granulocyte colony-stimulating factor (G-CSF), and granulocyte-macrophage colony-stimulating factor (GM-CSF). In a preferred embodiment disclosed therein, the stroma-free, serum-free, and chemically defined medium was composed of IMDM supplemented with a serum substitute consisting of 4 g/L BSA, 0.71 μg/mL insulin and 27.81 μg/mL transferrin, and a cytokine formula consisting of 5.53 ng/mL TPO, 2.03 ng/mL IL-3, 16 ng/mL SCF, 2.36 ng/mL IL-6, 4.43 ng/mL FL, 1.56 ng/mL GM-CSF, 2.64 ng/mL SCGF, 0.69 ng/mL IL-11, and 1.91 ng/mL G-CSF.

In a previous study, the applicants systematically developed an Mk medium containing IMDM supplemented 10% fetal bovine serum (FBS) to generate Mks from CD34⁺ cells. Factorial design and steepest ascent (SA) method were used to screen and optimize the effective cytokines (10.2 ng/mL TPO, 4.3 ng/mL IL-3, 15.0 ng/mL SCF, 5.6 ng/mL IL-6, 2.8 ng/mL FL, 2.8 ng/mL IL-9, and 2.8 ng/mL GM-CSF) in the Mk medium that facilitated ex vivo megakaryopoiesis from CD34⁺ cells (Te-Wei Chen et al. (2009), Biochemical and Biophysical Research Communications, 378:112-117).

While the Mk medium could facilitate the ex vivo megakaryopoiesis of human CD34⁺ cells, serum is a potential source of bacterial, mycoplasma, and viral contamination. Therefore, the applicants endeavored to develop a serum-free medium for the ex vivo production of megakaryocytes from human CD34⁺ cells.

### SUMMARY OF THE INVENTION

Therefore, according to a first aspect, this invention provides a cultivating medium for the ex vivo production of megakaryocytes from human CD34⁺ cells, the medium consisting essentially of a basal medium, a serum substitute and a cytokine cocktail, wherein:

the serum substitute consists essentially of human serum albumin, insulin, and transferrin; and

the cytokine cocktail consists essentially of thrombopoietin (TPO), stem cell factor (SCF), Flt-3 ligand (FL), interleukin-3 (IL-3), IL-6, IL-9, and granulocyte-macrophage colony-stimulating factor (GM-CSF).

In a second aspect, this invention provides a process for the ex vivo production of megakaryocytes from human CD34⁺ cells, comprising:

cultivating a population of human CD34⁺ cells in a cultivating medium consisting essentially of a basal medium, a serum substitute and a cytokine cocktail, wherein:
the serum substitute consists essentially of human serum albumin, insulin, and transferrin; and
the cytokine cocktail consists essentially of thrombopoietin (TPO), stern cell factor (SCF), Flt-3 ligand (FL), interleukin-3 (IL-3), IL-6, IL-9, and granulocyte-macrophage colony-stimulating factor (GM-CSF); and

harvesting a population of megakaryocytes thus formed from the cultivated population of human CD34⁺ cells.

### BRIEF DESCRIPTION OF THE DRAWING

The above and other objects, features and advantages of this invention will become apparent with reference to the following detailed description and the preferred embodiments taken in conjunction with the accompanying drawings, in which:

FIG. 1 is a bar diagram showing the number of Mks generated from serum-free expanded CD34⁺ cells after induction with different media for 1 week, in which serum-free expanded CD34⁺ cells were separately incubated in IMDM (the blank control), the SF-Mk medium as established in Example 1, infra, IMDM+10% fetal bovine serum (FBS), IMDM+2% FBS, Panserin 401, X-VIVO 10, X-VIVO 15, X-VIVO 20, Pro293, DMEM, RPMI 1640 medium, α-MEM, BME medium, F-12K medium, and Medium 199, wherein each of IMDM+10% FBS, IMDM+2% FBS, Panserin 401, X-VIVO 10, X-VIVO 15, X-VIVO 20, Pro293, DMEM, RPMI 1640 medium, α-MEM, BME medium, F-12K medium and Medium 199 was supplemented with a cytokine cocktail (CC) as screened in Example 1, infra; each bar is expressed as means±SD; and the symbol “***” indicates that p-value is less than 0.001;

FIG. 2 is a bar diagram showing the Mk count of each group after induction with different media for 1 week, in which serum-free expanded CD34⁺ cells were incubated in IMDM supplemented with 10% FBS and a reference cytokine cocktail as reported in Te-Wei Chen et al. (2009), supra (the FBS group), in IMDM supplemented with the reference cytokine cocktail and a serum substitute formula (4.9 g/L BSA, 2.72 μg/mL insulin, and 80 μg/mL transferrin)(the BSA group), and in the SF-Mk medium (the HSA group), respectively, each bar being expressed as means±SD (n=4);

FIG. 3 shows two culture strategies I and II for comparison of the ex vivo megakaryocytopoietic potential of CD34⁺ cells with and without subjection to serum-free expansion, in which in strategy I, CD34⁺ cells isolated from human umbilical cord blood (UCB) were incubated in the SF-HSC medium as reported in Chao-Ling Yao et al., (2003), supra, for 1 week and then in the SF-Mk medium for 2 weeks; and in strategy II, CD34⁺ cells isolated from UCB were incubated in the SF-Mk medium for 3 weeks;

FIG. 4 shows the cell surface antigen expression of cells generated from CD34⁺ cells via the culture strategies I and II of FIG. 3, as analyzed by flow cytometry and displayed by dot plots, in which panel i: freshly isolated CD34⁺ cells (week 0) labeled with FITC-CD41a and PE-CD34; panel ii: freshly isolated CD34⁺ cells (week 0) labeled with FITC-mouse IgG₁ and PE-mouse IgG₁ as isotype control; panel iii: cells cultured via strategy I at weeks 1, 2, and 3 and labeled with FITC-CD41a and PE-CD34, respectively; panel iv: cells cultured via strategy II at weeks 1, 2, and 3 and labeled with FITC-CD41a and PE-CD34, respectively; panel v: freshly isolated CD34⁺ cells (week 0) labeled with FITC-CD41a and PE-CD61; panel vi: freshly isolated CD34⁺ cells (week 0) labeled with FITC-mouse IgG₁ and PE-mouse IgG₁ as isotype control; panel vii: cells cultured via strategy I at weeks 1, 2, and 3 and labeled with FITC-CD41a and PE-CD61, respectively; and panel viii: cells cultured via strategy II at weeks 1, 2, and 3 and labeled with FITC-CD41a and PE-CD61, respectively; and the percentage value shown in each quadrant of a dot plot represents the percentage of total cells that fall within said quadrant;

FIG. 5 shows the growth kinetics of accumulated cells as generated from CD34⁺ cells via the culture strategies I and II of FIG. 3 (strategy I: black square, and strategy II: white square; n=4) at weeks 0, 1, 2, and 3, respectively, in which panel A, total nucleated cell (TNC); panel B, CD41a⁺CD34⁺ cell; and panel C, Mk; and the symbols “*”, “**”, and “***” indicate that p-values are less than 0.05, 0.01, and 0.001, respectively, as compared between said two culture strategies;

FIG. 6 shows the DNA ploidy distribution of Mks as displayed by dot plots, in which the Mks respectively collected after culturing CD34⁺ cells via the two culture strategies I and II of FIG. 3 at weeks 1 and 2 were subjected to flow cytometry analysis using FITC-CD41a labeling, followed by DNA content analysis using propidium iodide (PI) staining; and each of the percentage values shown in the dot plots represents the percentage of total cells that have a corresponding DNA ploidy as indicated therein;

FIG. 7 shows the mRNA expression of two megakaryocyte-lineage transcription factors, i.e., nuclear factor erythroid-derived 2 (NF-E2) and GATA-1, in cells generated from CD34⁺ cells via the two culture strategies I and II of FIG. 3 at weeks 0, 1, 2, and 3, respectively, in which the generated cells were analyzed by reverse transcription polymerase chain reaction (RT-PCR) (n=3) using glyceraldehyde-3-phosphate dehydrogenase (GAPDH) as an internal control;

FIG. 8 shows the cell surface antigen expression of CD41a⁺ cells before and after stimulation with a platelet activating reagent, as displayed by dot plots, in which the CD41a⁺ cells collected after culturing CD34⁺ cells via the culture strategies I (upper three panels) and II (lower three panels) of FIG. 3 at week 2 were subjected to flow cytometry analysis using FITC-CD41a and PE-CD62P labeling, and CD41a⁺ cells labeled with FITC-CD41a and PE-mouse IgG₁ were used as an isotype control; and the percentage value shown in each quadrant of a dot plot represents the percentage of total cells that fall within said quadrant;

FIG. 9 is a bar diagram which shows that after stimulation with a platelet activating reagent, CD62P is significantly upregulated in CD41a⁺ cells collected after culturing CD34⁺ cells via the culture strategies I and II of FIG. 3 at week 2, in which the data are expressed as means±SD; and the symbols “*” and “***” indicate that p-values are less than 0.05 and 0.001, respectively;

FIG. 10 shows the detection of human platelets (human CD61⁺ cells) in the peripheral blood (PB) of irradiated non-obese diabetic/severe combined immunodeficient (NOD/SCID) mice at day 9, day 11, and day 14 after transplantation, in which four groups of irradiated NOD/SCID mice (n=8 for each group) were injected via the tail vein with: Group 1, Dulbecco's phosphate-buffered saline (D-PBS) as a negative control; Group 2, 5×10⁵ serum-free expanded human CD34⁺ cells (strategy I, week 1) at day 0; Group 3, 5×10⁵ serum-free generated human CD61⁺ cells (strategy I, week 2) at day 0; and Group 4, 5×10⁵ serum-free expanded human CD34⁺ cells (strategy I, week 1) at day 0 and then 5×10⁵ serum-free generated human CD61⁺ cells (strategy I, week 2) from the same donor at day 7; the human platelets in the NOD/SCID mice's PB at day 9, day 11, and day 14 after transplantation were detected by flow cytometry using human CD-61-PE staining; the cell size of the detected human platelets is represented by the intensity in forward scatter (FSC); and the percentage value shown in a quadrant of a dot plot represents the percentage of total cells that fall within said quadrant;

FIG. 11 shows the kinetic analysis of human platelet production in irradiated NOD/SCID mice (human platelet/pt mouse PB), in which four groups of irradiated NOD/SCID mice (n=8 for each group) were injected via the tail vein with: Group 1, Dulbecco's phosphate-buffered saline (D-PBS) as a negative control; Group 2, 5×10⁵ serum-free expanded human CD34⁺ cells (strategy I, week 1) at day 0; Group 3, 5×10⁵ serum-free generated human CD61⁺ cells (strategy I, week 2) at day 0; and Group 4, 5×10⁵ serum-free expanded human CD34⁺ cells (strategy I, week 1) at day 0 and then 5×10⁵ serum-free generated human CD61⁺ cells (strategy I, week 2) from the same donor at day 7; the number of the human platelets in the NOD/SCID mice's PB at day 9, day 11, and day 14 after transplantation was counted by Sysmex KX-21 N cell count (Sysmex Corporation, Hamburg, Germany); and the data are expressed as means±SD; and

FIG. 12 shows the representative flow cytometry analysis of human Mks in the bone marrow (BM) of irradiated NOD/SCID mice at day 14 after transplantation, in which four groups of irradiated NOD/SCID mice (n=8 for each group) were injected via the tail vein with: Group 1, Dulbecco's phosphate-buffered saline (D-PBS) as a negative control; Group 2, 5×10⁵ serum-free expanded human CD34⁺ cells (strategy I, week 1) at day 0; Group 3, 5×10⁵ serum-free generated human CD61⁺ cells (strategy I, week 2) at day 0; and Group 4, 5×10⁵ serum-free expanded human CD34⁺ cells (strategy I, week 1) at day 0 and then 5×10⁵ serum-free generated human CD61⁺ cells (strategy I, week 2) from the same donor at day 7; the BM collected from NOD/SCID mice sacrificed at day 14 after transplantation was subjected to flow cytometry analysis by staining with human CD45-FITC for human leukocyte detection and human CD61-PE for human Mk detection; and the percentage value shown in a quadrant of a dot plot represents the percentage of total cells that fall within said quadrant.

### DETAILED DESCRIPTION OF THE INVENTION

It is to be understood that, if any prior art publication is referred to herein, such reference does not constitute an admission that the publication forms a part of the common general knowledge in the art, in Taiwan or any other country.

Unless otherwise defined, all technical and scientific terms used herein have the meaning commonly understood by a person skilled in the art to which this invention belongs. One skilled in the art will recognize many methods and materials similar or equivalent to those described herein, which could be used in the practice of this invention. Indeed, this invention is in no way limited to the methods and materials described.

As used herein, the transitional phrases “comprising,” “consisting essentially of” and “consisting of” define the scope of the appended claims with respect to what un-recited additional components, if any, are excluded from the scope of the claim. The term “comprising” is intended to be inclusive or open-ended and does not exclude additional, un-recited elements or method steps. The phrase “consisting of” excludes any element, step, or ingredient not specified in the claim. The phrase “consisting essentially of” limits the scope of a claim to the specified materials or steps and those that do not materially affect the basic and novel characteristic(s) of the claimed invention. All compositions or formulations identified herein can, in alternate embodiments, be more specifically defined by any of the transitional phrases “comprising,” “consisting essentially of” and “consisting of.”

Thrombocytopenia has been observed in patients after high-dose chemotherapy or hepatitis virus-related cirrhosis. Post-thrombocytopenia appears particularly frequently in cancer patients after they receive CB HSC transplantation. Transfusion of ex vivo expanded Mks is a new promising strategy for accelerating Mk and platelet recovery after CB transplantation. However, there has yet to be established a feasible approach to generate large amounts of Mks under serum-free conditions.

In this invention, in order to massively produce megakaryocytes from human CD34⁺ cells, the applicants developed a stromal-free, serum-free, and cytokine-optimized medium by using a two-level factorial design and the steepest ascent (SA) method in combination. Specifically, three serum substitutes, i.e., human serum albumin (HSA), insulin, and transferrin, and seven kinds of cytokines, i.e., thrombopoietin (TPO), stem cell factor (SCF), Flt-3 ligand (FL), interleukin-3 (IL-3), IL-6, IL-9, and granulocyte-macrophage colony-stimulating factor (GM-CSF), were selected by two-level fractional factorial design and their concentrations were optimized using a SA path for Mk generation.

The applicants found that the developed medium could provide dual effects to the cultivated human CD34⁺ cells. Specifically, when the developed medium was used to cultivate human CD34⁺ cells, either freshly isolated from a newborn's cord blood or having been subcultured in an expansion medium after isolation from a newborn's cord blood, a large amount (expansion effect) of functional Mks (induction effect) could be generated. In addition, the generated Mks, as characterized by surface marker expression of CD41a and CD61, gene expression of NF-E2 and GATA-1, polyploidy distribution, and platelet activation ability, were proven to be effective in boosting platelet recovery in X-ray-irradiated NOD/SCID mice.

In contrast to commercial media or media from other reports, the developed medium has a low concentration of cytokines, low induction period, and high induction efficiency. Serum-free Mks generated in this manner may serve as an alternative Mk and platelet source for future clinical applications.

Accordingly, this invention provides a cultivating medium for the ex vivo production of megakaryocytes from human CD34⁺ cells, the medium consisting essentially of a basal medium, a serum substitute and a cytokine cocktail, wherein:

the serum substitute consists essentially of human serum albumin, insulin, and transferrin; and

the cytokine cocktail consists essentially of thrombopoietin (TPO), stem cell factor (SCF), Flt-3 ligand (FL), interleukin-3 (IL-3), IL-6, IL-9, and granulocyte-macrophage colony-stimulating factor (GM-CSF).

This invention also provides a process for the ex vivo production of megakaryocytes from human CD34⁺ cells, comprising:

cultivating a population of human CD34⁺ cells in a cultivating medium consisting essentially of a basal medium, a serum substitute and a cytokine cocktail, wherein:
the serum substitute consists essentially of human serum albumin, insulin, and transferrin; and
the cytokine cocktail consists essentially of thrombopoietin (TPO), stem cell factor (SCF), Flt-3 ligand (FL), interleukin-3 (IL-3), IL-6, IL-9, and granulocyte-macrophage colony-stimulating factor (GM-CSF); and

harvesting a population of megakaryocytes thus formed from the cultivated population of human CD34⁺ cells.

According to this invention, the population of human CD34⁺ cells is any one of the following:
(i) a population of human CD34⁺ cells freshly isolated from a newborn's cord blood; and
(ii) a population of human CD34⁺ cells that have been subcultured in an expansion medium after isolation from a newborn's cord blood.

According to this invention, the newborn's cord blood may be collected from the postpartum placenta and/or the umbilical cord. In a preferred embodiment of this invention, the newborn's cord blood is collected from the umbilical cord.

According to this invention, human CD34⁺ cells freshly isolated from a newborn's cord blood may be subjected to cultivation in a medium effective to expand hematopoletic stem cells, in particular CD34⁺ cells. Such expansion media have been reported in various literatures, including those reported in the applicants' earlier publications. In a preferred embodiment of this invention, the SF-HSC medium as reported in Chao-Ling Yao et al. (2003), supra, serves as the expansion medium.

The basal medium suitable for use in this invention may be selected from the group consisting of an Iscove's modified Dulbecco's medium (IMDM), a Dulbecco's modified Eagle's medium (DMEM), a RPMI 1640 medium, a minimum essential medium alpha medium (α-MEM), a basal medium Eagle medium (BME medium), an F-12K nutrient mixture medium (F-12K medium), and a Medium 199. In a preferred embodiment of this invention, the basal medium is an Iscove's modified Dulbecco's medium (IMDM).

According to this invention, the human serum albumin in the serum substitute may be present at a concentration ranging from 4.0 to 32.0 g/L, and preferably from 7.0 to 22.0 g/L, based on the volume of the cultivating medium. In a preferred embodiment of this invention, the human serum albumin in the serum substitute is present at a concentration of 8 g/L.

According to this invention, the insulin in the serum substitute may be present at a concentration ranging from 0.9 to 7.2 μg/mL, and preferably from 1.6 to 4.9 μg/mL, based on the volume of the cultivating medium. In a preferred embodiment of this invention, the insulin in the serum substitute is present at a concentration of 1.8 μg/mL.

According to this invention, the transferrin in the serum substitute may be present at a concentration ranging from 25.3 to 202.0 μg/mL, and preferably from 44.2 to 138.9 μg/mL, based on the volume of the cultivating medium. In a preferred embodiment of this invention, the transferrin in the serum substitute is present at a concentration of 50.5 μg/mL.

According to this invention, the TPO in the cytokine cocktail may be present at a concentration ranging from 1.8 to 13.2 ng/mL, and preferably from 2.4 to 5.4 ng/mL, based on the volume of the cultivating medium. In a preferred embodiment of this invention, the TPO in the cytokine cocktail is present at a concentration of 3.0 ng/mL.

According to this invention, the SCF in the cytokine cocktail is present at a concentration ranging from 7.5 to 55.0 ng/mL, and preferably from 10.0 to 22.5 ng/mL, based on the volume of the cultivating medium. In a preferred embodiment of this invention, the SCF in the cytokine cocktail is present at a concentration of 12.5 ng/mL.

According to this invention, the FL in the cytokine cocktail may be present at a concentration ranging from 0.8 to 6.1 ng/mL, and preferably from 1.1 to 2.5 ng/mL, based on the volume of the cultivating medium. In a preferred embodiment of this invention, the FL in the cytokine cocktail is present at a concentration of 1.4 ng/mL.

According to this invention, the IL-3 in the cytokine cocktail may be present at a concentration ranging from 1.7 to 12.7 ng/mL, and preferably from 2.3 to 5.2 ng/mL, based on the volume of the cultivating medium. In a preferred embodiment of this invention, the IL-3 in the cytokine cocktail is present at a concentration of 2.9 ng/mL.

According to this invention, the IL-6 in the cytokine cocktail may be present at a concentration ranging from 0.3 to 2.1 ng/mL, and preferably from 0.4 to 0.9 ng/mL, based on the volume of the cultivating medium. In a preferred embodiment of this invention, the IL-6 in the cytokine cocktail is present at a concentration of 0.5 ng/mL.

According to this invention, the IL-9 in the cytokine cocktail may be present at a concentration ranging from 1.0 to 7.5 ng/mL, and preferably from 1.4 to 3.1 ng/mL, based on the volume of the cultivating medium. In a preferred embodiment of this invention, the IL-9 in the cytokine cocktail is present at a concentration of 1.7 ng/mL.

According to this invention, the GM-CSF in the cytokine cocktail is present at a concentration ranging from 4.4 to 32.1 ng/mL, and preferably from 5.8 to 13.1 ng/mL, based on the volume of the cultivating medium. In a preferred embodiment of this invention, the GM-CSF in the cytokine cocktail is present at a concentration of 7.3 ng/mL.

In a preferred embodiment of this invention, the cultivating medium consists essentially of a basal medium, a serum substitute and a cytokine cocktail, wherein:

the basal medium is an Iscove's modified Dulbecco's medium;

the serum substitute consists essentially of human serum albumin, insulin, and transferrin, wherein based on the volume of the cultivating medium, the human serum albumin is present at a concentration ranging from 4.0 to 32.0 g/L, the insulin is present at a concentration ranging from 0.9 to 7.2 μg/mL, and the transferrin is present at a concentration ranging from 25.3 to 202.0 μg/mL; and

the cytokine cocktail consists essentially of thrombopoietin, stem cell factor, Flt-3 ligand, interleukin-3, interleukin-6, interleukin-9, and granulocyte-macrophage colony-stimulating factor, wherein based on the volume of the cultivating medium, the thrombopoietin is present at a concentration ranging from 1.8 to 13.2 ng/mL, the stem cell factor is present at a concentration ranging from 7.5 to 55.0 ng/mL, the Flt-3 ligand is present at a concentration ranging from 0.8 to 6.1 ng/mL, the interleukin-3 is present at a concentration ranging from 1.7 to 12.7 ng/mL, the interleukin-6 is present at a concentration ranging from 0.3 to 2.1 ng/mL, the interleukin-9 is present at a concentration ranging from 1.0 to 7.5 ng/mL, and the granulocyte-macrophage colony-stimulating factor is present at a concentration ranging from 4.4 to 32.1 ng/mL.

In a more preferred embodiment of this invention, the cultivating medium consists essentially of a basal medium, a serum substitute and a cytokine cocktail, wherein:

the basal medium is an Iscove's modified Dulbecco's medium;

the serum substitute consists essentially of human serum albumin, insulin, and transferrin, wherein based on the volume of the cultivating medium, the human serum albumin is present at a concentration ranging from 7.0 to 22.0 g/L, the insulin is present at a concentration ranging from 1.6 to 4.9 μg/mL, and the transferrin is present at a concentration ranging from 44.2 to 138.9 μg/mL; and

the cytokine cocktail consists essentially of thrombopoietin, stem cell factor, Flt-3 ligand, interleukin-3, interleukin-6, interleukin-9, and granulocyte-macrophage colony-stimulating factor, wherein based on the volume of the cultivating medium, the thrombopoietin is present at a concentration ranging from 2.4 to 5.4 ng/mL, the stem cell factor is present at a concentration ranging from 10.0 to 22.5 ng/mL, the Flt-3 ligand is present at a concentration ranging from 1.1 to 2.5 ng/mL, the interleukin-3 is present at a concentration ranging from 2.3 to 5.2 ng/mL, the interleukin-6 is present at a concentration ranging from 0.4 to 0.9 ng/mL, the interleukin-9 is present at a concentration ranging from 1.4 to 3.1 ng/mL, and the granulocyte-macrophage colony-stimulating factor is present at a concentration ranging from 5.8 to 13.1 ng/mL.

In a more further preferred embodiment of this invention, the cultivating medium consists essentially of a basal medium, a serum substitute and a cytokine cocktail, wherein the basal medium is an Iscove's modified Dulbecco's medium; and based on the volume of the cultivating medium, the serum substitute consists essentially of 8 g/L human serum albumin, 1.8 μg/mL insulin and 50.5 μg/mL transferrin, and the cytokine cocktail consists essentially of 3.0 ng/mL thrombopoietin, 12.5 ng/mL stem cell factor, 1.4 ng/mL Flt-3 ligand, 2.9 ng/mL interleukin-3, 0.5 ng/mL interleukin-6, 1.7 ng/mL interleukin-9 and 7.3 ng/mL granulocyte-macrophage colony-stimulating factor.

This invention will be further described by way of the following examples. However, it should be understood that the following examples are solely intended for the purpose of illustration and should not be construed as limiting the invention in practice.

### EXAMPLES

### Reagents

The following recombinant human cytokines and chemicals were used: thrombopoietin (TPO), stem cell factor (SCF), interleukin-3 (IL-3), IL-6, IL-9, Flt-3 ligand (FL) and granulocyte-macrophage colony-stimulating factor (GM-CSF) were purchased from PeproTech EC Ltd. (London, UK); human serum albumin (HSA) was purchased from ZLB Behring GmbH (Marburg, Germany); bovine serum albumin (BSA) and human insulin were purchased from Sigma (St. Louis, Mo., USA); human and 2-mercaptoethanol (2-ME) were obtained from GIBCO (Carlsbad, Calif., USA); and fetal bovine serum (FBS) was purchased from HyClone (Logan, Utah, USA).

The following commercial serum-free media were used: Iscove's modified Dulbecco's medium (IMDM) was purchased from HyClone (Logan, Utah, USA); X-VIVO 10, X-VIVO 15, and X-VIVO 20 were purchased from BioWhittaker (Walkersville, Md., USA); Panserin 401 and Pro 293 were purchased from Pan Biotech GmbH (Aidenbach, Germany) and GIBCO, respectively; and Dulbecco's modified Eagle's medium (DMEM), RPMI 1640 medium, minimum essential medium alpha medium (α-MEM), basal medium Eagle medium (BME medium), F-12K nutrient mixture medium (F-12K medium), and Medium 199 were all purchased from Gibco BRL (Grand Island, N.Y., USA).

The following fluorescein isothiocyanate (FITC)- or phycoerythrin (PE)-conjugated monoclonal antibodies were used: anti-human CD34-FITC, CD45-FITC, CD34-PE and CD61-PE were purchased from Miltenyi Biotec (Bergisch Gladbach, Germany); and anti-human CD41a-FITC, CD62P-PE, and mouse IgG₁, either FITC- or PE-conjugated, were purchased from eBioscience (San Diego, Calif., USA).

### General Experimental Procedures:

1. Isolation of CD34⁺ Cells from Human Umbilical Cord Blood:

With approval from the scientific committees of the Food Industry Research and Development Institute (Hsinchu, Taiwan), human umbilical cord blood (UCB) was collected and processed according to governmental regulations (“Guidelines for collection and use of human specimens for research,” Department of Health, Taiwan). In addition, informed consent was obtained from laboring mothers for donation of UCB. Briefly, the UCB samples were harvested from healthy full-term newborns (delivered by either an uneventful vaginal birth or a cesarean section) using a standard 250 mL blood bag containing citrate-phosphate-dextrose-adenine anticoagulant (Terumo, Shibuya-ku, Tokyo, Japan). Mononuclear cells (MNCs) were subsequently isolated from the UCB samples by Ficoll-Paque (Amersham Biosciences, Uppsala, Sweden) density gradient centrifugation within 24 hrs. Thereafter, fresh CD34⁺ cells were purified with CD34 microbeads by a Miltenyi VarioMACS device (Miltenyi Biotec, Bergisch Gladbach, Germany) according to the manufacturer's instructions.

### 2. Serum-Free Expansion of CD34⁺ Cells:

Serum-free expansion of CD34⁺ cells was implemented substantially according to the procedures as set forth in Chao-Ling Yao et al., (2003), supra, except for minor modifications. Briefly, fresh CD34⁺ cells were seeded at 5×10⁴ cells/mL in 24-well plates (Falcon, N.J., USA) in SF-HSC medium (1 mL per well). The SF-HSC medium was composed of IMDM supplemented with a cytokine cocktail (8.5 ng/mL TPO, 4.1 ng/mL IL-3, 15 ng/mL SCF, 6.7 ng/mL FL, 0.8 ng/mL IL-6, 3.2 ng/mL G-CSF, and 1.3 ng/mL GM-CSF) and serum substitutes (1.5 g/L BSA, 4.4 μg/mL insulin, 60 μg/mL transferrin, and 25.9 μM 2-ME). Prior to use in the following assays, the CD34⁺ cells were cultivated with change of medium at weekly intervals, in which the cell density was readjusted to 5×10⁴ cells/mL.

Calculation of the cell number was conducted by total nucleated cell count (TNC count), in which the collected cells were treated with ZAP-OGLOBIN II Lytic Reagent (Beckman Coulter Inc., Fullerton, Calif., USA) for 3 minutes to cause lysis of RBCs, followed by counting the number of cells having a size ranging from 4 to 12 μm on a Coulter counter model Z1 (Coulter Electronics Ltd., Beds, UK). Each experiment was repeated at least four times.

### 3. Two-Level Factorial Design and Steepest Ascent (SA) Method:

A factorial design and a SA method were combined to determine optimal serum substitutes and cytokines as well as their concentrations for ex vivo induction of megakaryocytes (Mks, CD41a⁺CD61⁺ cells) from CD34⁺ cells (Box GEP. et al. (1978), Statistics for Experimenters: An Introduction to Design, Data Analysis, and Model Building, Wiley, New York). In addition, the statistical significance was determined by an F-test, and the significance of the regression coefficient was analyzed by a t-test. Briefly, factorial design data were regressed by Design Expert statistical software (Stat-Ease Inc., Minneapolis, Minn.) to obtain a polynomial function represented by a simplified equation (1) as shown below:

CD41a⁺CD61⁺ cells(cells/mL)=α₀+Σαᵢxᵢ, (1)

wherein as are the fitted constants and x's are coded variables for the tested cytokines or serum substitutes, and the coefficient αᵢ corresponds to the main effect.

In the experiments of this invention, the statistically significant main (p-value<0.05) terms were considered whereas the insignificant higher-order terms were neglected. The first-ordered equation could identify effective factors with positive coefficients, eliminate unnecessary factors with negative coefficients, and provide necessary information for development of the SA path to optimize the concentrations of cytokines and serum substitutes for ex vivo Mk induction from CD34⁺ cells.

The strategy for developing a SF-Mk medium according to this invention was as follows:
step 1): screening effective serum substitutes and optimizing their concentrations using IMDM as a serum-free basal medium;
step 2): screening an effective cytokine cocktail and optimizing cytokine concentrations using IMDM supplemented with effective serum substitutes; and
step 3): comparing the SF-Mk medium thus developed with different commercial serum-free media.

In the screening experiments, serum-free expanded CD34⁺ cells were seeded at a concentration of 5×10⁴ cells/mL in 24-well plates (1 mL per well) with a varied combination of serum substitutes or cytokines according to the experimental design. After cultivation for 1 week, the cells were collected and subjected to the TNC count as described above and a megakaryocyte count (Mk count) so as to evaluate the ex vivo megakaryocytopoietic effects of the tested serum substitutes and cytokines.

The Mk count was conducted by labeling the collected cells with CD41a-FITC and CD61-PE and analyzed according to the operating procedure described in the following section, entitled “4. Analysis of cell surface antigen.”

### 4. Analysis of Cell Surface Antigen:

Cell suspension containing about 10⁶ cells to be analyzed was dispensed in a polystyrene round-bottom tube (12×75 mm) (Falcon, N.J., USA) and was washed with FACS buffer [D-PBS containing 1% FBS and 0.5% sodium azide (NaN₃, Sigma)]. After centrifugation at 700×g for 5 minutes, the resultant supernatant was removed and the precipitated cells were incubated with a first antibody conjugated with FITC or PE in FACS buffer (1:10˜1:20) in the dark at 4° C. for 30 minutes. The incubation was terminated by washing three times with 1 mL FACS buffer. Optionally, the first antibody-labeled cells were further incubated with a second antibody conjugated with PE or FITC in the same manner. After washing with FACS buffer, the labeled cells were resuspended in 1 mL FACS buffer and analyzed on a FACSCalibur analyzer (Becton-Dickinson, NJ, USA). The data were analyzed by CellQuest software. A replicate sample incubated with FITC- or PE-conjugated mouse IgG₁ was used as an isotype control for specificity.

### 5. Statistical Analysis:

The experimental results from multiple independent experiments were expressed as mean±standard deviation (SD) and were evaluated by the paired samples t-test. A p-value less than 0.05 was considered to be statistically significant. The symbols “*,” “**,” and “***” indicate that the p-values of the comparison are less than 0.05, 0.01, and 0.001, respectively.

### Example 1

### Screening of Serum Substitutes and Cytokines Optimal for the Ex Vivo Production of Megakaryocytes from Serum-Free Expanded CD34⁺ Cells

### A. Screening and Optimization of Serum Substitutes:

Based on an extensive review and the applicants' experimental experiences, HSA, insulin, transferrin, 2-ME, L-glutamine, and sodium pyruvate were selected for the ex vivo production of megakaryocytes from serum-free expanded CD34⁺ cells. In a preliminary screening, HSA, insulin, and transferrin were observed to have significantly positive effects on ex vivo megakaryocytopoiesis of the serum-free expanded CD34⁺ cells, whereas 2-ME, L-glutamine, and sodium pyruvate were observed to have no effect (data not shown).

According to the preliminary screening results, the applicants adopted 2³ full factorial design (eight trials with complete degrees of freedom) to evaluate the effect of HSA, insulin and transferrin on the ex vivo megakaryocytopoiesis of the serum-free expanded CD34⁺ cells in IMDM supplemented with a reference cytokine cocktail as previously reported in Te-Wei Chen et al. (2009), supra. Design matrix of the 2³ full factorial design and the TNC count and Mk count after 1-week induction are shown in Table 1.

TABLE 1
Design matrix of the 2³ full factorial design and the TNC count and Mk
count after 1-week induction^{a}

| | HSA | insulin | transferrin | TNC count^{b} | Mk count^{c} |
|---|---|---|---|---|---|
| Trial | (15 g/L) | (0.01 g/L) | (0.15 g/L) | (10⁶/mL) | (10⁵/mL) |
| 1 | −1 | −1 | −1 | 0.01 | 0.19 |
| 2 | +1 | −1 | −1 | 0.62 | 0.80 |
| 3 | −1 | +1 | −1 | 0.16 | 0.15 |
| 4 | +1 | +1 | −1 | 0.86 | 1.50 |
| 5 | −1 | −1 | +1 | 0.44 | 0.66 |
| 6 | +1 | −1 | +1 | 0.65 | 0.96 |
| 7 | −1 | +1 | +1 | 0.47 | 0.68 |
| 8 | +1 | +1 | +1 | 0.84 | 1.59 |

^{a}The experiment was repeated 4 times.
^{b}TNC count -- expressed as mean.
^{c}Mk count -- expressed as mean.
+1: Addition of an indicated amount of the tested serum substitute.
−1: No addition.

A first-order model was regressed according to the data shown in Table 1 and is represented by the following equation (2):

Megakaryocytes/mL(×10⁴)=9.07+3.98x₁+2.21x₂+4.34x₃ (2)

in which:

x₁=coded variable for HSA;

x₂=coded variable for insulin; and

x₃=coded variable for transferrin.

Equation (2) specified that HSA, insulin, and could promote Mk generation from serum-free expanded CD34⁺ cells. Then, a SA path was determined from the coefficients in equation (2) to obtain optimal concentration of each of the tested serum substitutes for the maximal Mk generation. The concentration of each of the 3 tested serum substitutes along the SA path and the TNC count and Mk count after 1-week induction are summarized in Table 2.

TABLE 2
Concentration of each of the tested serum substitutes along the SA
path and the TNC count and Mk count after 1-week induction^{a}

| | HSA | insulin | transferrin | TNC count^{b} | Mk count^{c} |
|---|---|---|---|---|---|
| Step | (g/L) | (μg/mL) | (μg/mL) | (10⁶/mL) | (10⁵/mL) |
| 1 | 1.0 | 0.2 | 6.3 | 0.42 ± 0.02 | 1.41 ± 0.22 |
| 2 | 2.0 | 0.5 | 12.6 | 0.68 ± 0.04 | 1.71 ± 0.07 |
| 3 | 3.0 | 0.7 | 18.9 | 0.77 ± 0.14 | 1.79 ± 0.08 |
| 4 | 4.0 | 0.9 | 25.3 | 0.85 ± 0.17 | 2.12 ± 0.21 |
| 5 | 5.0 | 1.1 | 31.6 | 1.02 ± 0.03 | 2.42 ± 0.08 |
| 6 | 6.0 | 1.4 | 37.9 | 1.05 ± 0.07 | 2.24 ± 0.18 |
| 7 | 7.0 | 1.6 | 44.2 | 1.27 ± 0.08 | 2.57 ± 0.01 |
| 8 | 8.0 | 1.8 | 50.5 | 1.42 ± 0.04 | 3.01 ± 0.29 |
| 9 | 9.0 | 2.0 | 56.8 | 1.48 ± 0.07 | 2.70 ± 0.25 |
| 10 | 10.0 | 2.2 | 63.1 | 1.42 ± 0.09 | 2.58 ± 0.10 |
| 11 | 11.0 | 2.5 | 69.4 | 1.43 ± 0.03 | 2.60 ± 0.05 |
| 12 | 12.0 | 2.7 | 75.7 | 1.46 ± 0.01 | 2.61 ± 0.34 |
| 13 | 22.0 | 4.9 | 138.9 | 1.40 ± 0.01 | 2.56 ± 0.18 |
| 14 | 32.0 | 7.2 | 202.0 | 1.06 ± 0.03 | 2.05 ± 0.34 |
| 15 | 42.0 | 9.4 | 265.1 | 0.85 ± 0.04 | 1.89 ± 0.07 |
| 16 | 52.0 | 11.7 | 328.2 | 0.68 ± 0.05 | 1.71 ± 0.20 |

^{a}The experiment was repeated 4 times.
^{b}TNC count -- expressed as mean ± SD.
^{c}Mk count -- expressed as mean ± SD.

As shown in Table 2, the Mk count initially increased along the SA path, reaching its maximum (3.01±0.29×10⁵ cells/mL) at step 8. After step 8, Mk count declined gradually. Consequently, a serum substitute having a formula of 8 g/L HSA, 1.8 μg/mL insulin and 50.5 μg/mL transferrin was optimized and referred to as “HIT” hereinafter.

### B. Screening and Optimization of Cytokines:

Synergistic or inhibitory interactions of cytokines are complex and crucial to the megakaryocytopoiesis process. In a previous study, the applicants found that TPO, IL-3, SCF, IL-6, FL, IL-9, and GM-CSF were necessary for the ex vivo megakaryocytopoiesis of serum-free expanded CD34⁺ cells under serum-containing conditions (Te-Wei Chen et al. (2009), supra). In this example, the applicants identified the effects of these 7 cytokines on the ex vivo megakaryocytopoiesis under serum-free conditions by a 2⁷⁻³ fractional factorial design (16 trials with sufficient degrees of freedom). The experiment was essentially conducted in accordance with the operating procedures as set forth in the preceding section entitled “A. Screening and optimization of serum substitutes,” except that the culture medium used herein was IMDM supplemented with the HIT as screened above and variable cytokines. Design matrix of the 2⁷⁻³ fractional factorial design and TNC count and Mk count after 1-week induction are shown in Table 3.

TABLE 3
Design matrix of the 2⁷⁻³ fractional factorial design and TNC count and
Mk count after 1-week induction^{a}

| | | | | | | | | TNC count^{b} | Mk count^{c} |
|---|---|---|---|---|---|---|---|---|---|
| Trial | TPO | IL-3 | SCF | IL-6 | FL | IL-9 | GM-CSF | (10⁶/mL) | (10⁵/mL) |
| 1 | −1 | −1 | −1 | −1 | −1 | −1 | −1 | 0.05 | 0.02 |
| 2 | +1 | −1 | −1 | −1 | +1 | −1 | +1 | 0.32 | 0.37 |
| 3 | −1 | +1 | −1 | −1 | +1 | +1 | −1 | 0.27 | 0.12 |
| 4 | +1 | +1 | −1 | −1 | −1 | +1 | +1 | 0.41 | 0.29 |
| 5 | −1 | −1 | +1 | −1 | +1 | +1 | +1 | 1.86 | 1.79 |
| 6 | +1 | −1 | +1 | −1 | −1 | +1 | −1 | 0.31 | 0.69 |
| 7 | −1 | +1 | +1 | −1 | −1 | −1 | +1 | 1.79 | 1.66 |
| 8 | +1 | +1 | +1 | −1 | +1 | −1 | −1 | 0.86 | 1.19 |
| 9 | −1 | −1 | −1 | +1 | −1 | +1 | +1 | 0.19 | 0.17 |
| 10 | +1 | −1 | −1 | +1 | +1 | +1 | −1 | 0.12 | 0.21 |
| 11 | −1 | +1 | −1 | +1 | +1 | −1 | +1 | 0.38 | 0.27 |
| 12 | +1 | +1 | −1 | +1 | −1 | −1 | −1 | 0.23 | 0.19 |
| 13 | −1 | −1 | +1 | +1 | +1 | −1 | −1 | 0.21 | 0.19 |
| 14 | +1 | −1 | +1 | +1 | −1 | −1 | +1 | 1.49 | 1.78 |
| 15 | −1 | +1 | +1 | +1 | −1 | +1 | −1 | 0.64 | 0.97 |
| 16 | +1 | +1 | +1 | +1 | +1 | +1 | +1 | 2.33 | 2.70 |

^{a}The experiment was repeated 4 times.
^{b}TNC count -- expressed as mean.
^{c}Mk count -- expressed as mean.
+1: The final concentration of the added cytokine in culture medium was 50 ng/mL.
−1: No addition.

A first-order model was regressed based on the data shown in Table 3 and is represented by the following equation (3):

Megakaryocytes/mL(×10⁴)=7.88+1.40x₁+1.35x₂+5.83x₃+0.22x₄+0.64x₅+0.80x₆+3.40x₇ (3)

in which:

x₁=coded variable for TPO;

x₂=coded variable for IL-3;

x₃=coded variable for SCF;

x₄=coded variable for IL-6;

x₅=coded variable for FL;

x₆=coded variable for IL-9; and

x₇=coded variable for GM-CSF.

A SA path was determined from the coefficients in equation (3) to obtain optimal concentration for each cytokine for the maximal MK generation. The concentration of each of the tested cytokines along the SA path and TNC count and Mk count after 1-week induction are summarized in Table 4.

TABLE 4
Concentration of each of the tested cytokines along the SA path and
TNC count and Mk count after 1-week induction^{a}

| | TPO | IL-3 | SCF | IL-6 | FL | IL-9 | GM-CSF | TNC count^{b} | Mk count^{c} |
|---|---|---|---|---|---|---|---|---|---|
| Step | (ng/mL) | (ng/mL) | (ng/mL) | (ng/mL) | (ng/mL) | (ng/mL) | (ng/mL) | (10⁶/mL) | (10⁵/mL) |
| 1 | 0.6 | 0.6 | 2.5 | 0.1 | 0.3 | 0.3 | 1.5 | 0.45 ± 0.12 | 1.57 ± 0.46 |
| 2 | 1.2 | 1.2 | 5.0 | 0.2 | 0.6 | 0.7 | 2.9 | 0.66 ± 0.29 | 1.85 ± 0.36 |
| 3 | 1.8 | 1.7 | 7.5 | 0.3 | 0.8 | 1.0 | 4.4 | 0.82 ± 0.28 | 2.28 ± 0.46 |
| 4 | 2.4 | 2.3 | 10.0 | 0.4 | 1.1 | 1.4 | 5.8 | 1.01 ± 0.31 | 2.52 ± 0.37 |
| 5 | 3.0 | 2.9 | 12.5 | 0.5 | 1.4 | 1.7 | 7.3 | 1.26 ± 0.23 | 2.92 ± 0.35 |
| 6 | 3.6 | 3.5 | 15.0 | 0.6 | 1.7 | 2.1 | 8.8 | 1.19 ± 0.24 | 2.61 ± 0.29 |
| 7 | 4.2 | 4.0 | 17.5 | 0.7 | 1.9 | 2.4 | 10.2 | 1.35 ± 0.32 | 2.76 ± 0.38 |
| 8 | 4.8 | 4.6 | 20.0 | 0.8 | 2.2 | 2.7 | 11.7 | 1.35 ± 0.27 | 2.73 ± 0.41 |
| 9 | 5.4 | 5.2 | 22.5 | 0.9 | 2.5 | 3.1 | 13.1 | 1.40 ± 0.21 | 2.59 ± 0.63 |
| 10 | 6.0 | 5.8 | 25.0 | 1.0 | 2.8 | 3.4 | 14.6 | 1.34 ± 0.16 | 2.46 ± 0.49 |
| 11 | 7.2 | 6.9 | 30.0 | 1.2 | 3.3 | 4.1 | 17.5 | 1.29 ± 0.22 | 2.15 ± 0.43 |
| 12 | 8.4 | 8.1 | 35.0 | 1.4 | 3.9 | 4.8 | 20.4 | 1.37 ± 0.20 | 2.44 ± 0.36 |
| 13 | 9.6 | 9.2 | 40.0 | 1.5 | 4.4 | 5.5 | 23.3 | 1.39 ± 0.17 | 2.37 ± 0.53 |
| 14 | 10.8 | 10.4 | 45.0 | 1.7 | 5.0 | 6.1 | 26.2 | 1.34 ± 0.16 | 2.35 ± 0.50 |
| 15 | 12.0 | 11.5 | 50.0 | 1.9 | 5.5 | 6.8 | 29.2 | 1.27 ± 0.18 | 2.23 ± 0.41 |
| 16 | 13.2 | 12.7 | 55.0 | 2.1 | 6.1 | 7.5 | 32.1 | 1.32 ± 0.21 | 2.28 ± 0.38 |

^{a}The experiment was repeated 4 times.
^{b}TNC count -- expressed as mean ± SD.
^{c}Mk count -- expressed as mean ± SD.

As shown in Table 4, Mk count initially increased along the SA path, reaching its maximum (2.92±0.35×10⁵ cells/mL) at step 5. Consequently, a cytokine cocktail was optimized to include 3.0 ng/mL TPO, 2.9 ng/mL IL-3, 12.5 ng/mL SCF, 0.5 ng/mL IL-6, 1.4 ng/mL FL, 1.7 ng/mL IL-9, and 7.3 ng/mL GM-CSF.

As a result, a serum-free megakaryocyte medium (referred to as “SF-Mk medium” hereinafter) composed of IMDM, HIT (8 g/L HSA, 1.8 μg/mL insulin, 50.5 μg/mL transferrin), and the cytokine cocktail (3.0 ng/mL TPO, 2.9 ng/mL IL-3, 12.5 ng/mL SCF, 0.5 ng/mL IL-6, 1.4 ng/mL FL, 1.7 ng/mL IL-9, and 7.3 ng/mL GM-CSF) was developed.

### Example 2

### Comparison of SF-Mk Medium with Commercially Available Serum-Free Media for Ex Vivo Megakaryocytopoietic Effect

To determine the ex vivo megakaryocytopoietic effect of the SF-Mk medium as established in the above Example 1, eleven commercially available serum-free media were used for comparison in terms of their performance on Mk generation.

Serum-free expanded CD34⁺ cells as obtained by the procedures set forth in Section “2. Serum-free expansion of CD34⁺ cells” of the General Experimental Procedures, were seeded into each well of a 24-well plate at a concentration of 5×10⁴ cells/well and were incubated in 1 mL of any one of the following media: SF-Mk as established in Example 1, IMDM+10% FBS, IMDM+2% FBS, Panserin 401, X-VIVO 10, X-VIVO 15, X-VIVO 20, Pro 293, DMEM, RPMI 1640, α-MEM, BME medium, F-12K medium, and Medium 199, respectively. IMDM+10% FBS, IMDM+2% FBS, Panserin 401, X-VIVO 10, X-VIVO 15, X-VIVO 20, Pro 293, DMEM, RPMI 1640, α-MEM, BME medium, F-12K medium and Medium 199 were respectively supplemented with the cytokine cocktail (CC) as screened in Example 1. For purpose of comparison, serum-free expanded CD34⁺ cells were incubated in IMDM only as a blank control. After 1-week induction, the Mk number of each group was calculated.

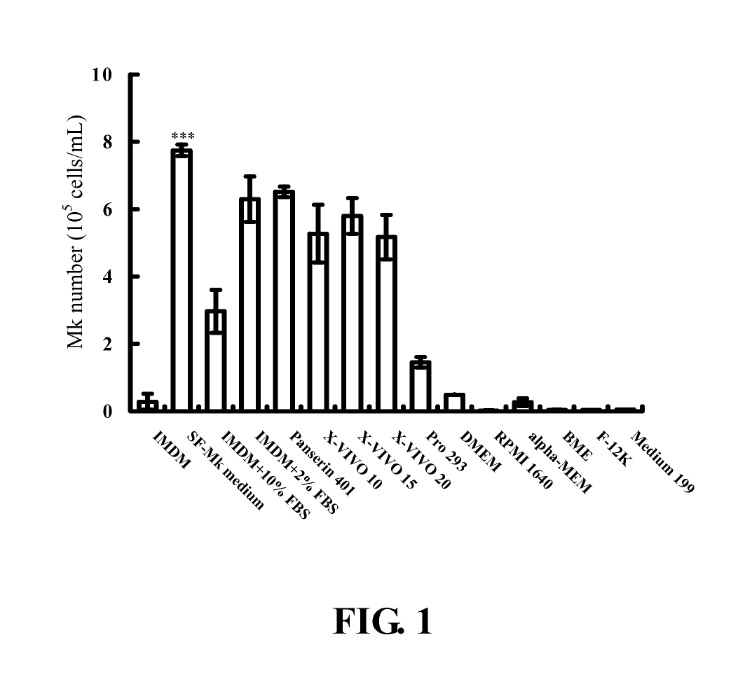

FIG. 1 is a bar diagram showing the number of Mks generated from serum-free expanded CD34⁺ cells after induction with different media for 1 week. It can be seen from FIG. 1 that the SF-Mk medium according to this invention exhibits the greatest ability on Mk generation as compared to the remaining tested media (p<0.001).

### Example 3

### Comparison of HSA, BSA and FBS Upon the Ex Vivo Megakaryocytopoiesis of Serum-Free Expanded CD34⁺ Cells

This example was performed to determine the influence of HSA, BSA and FBS upon the ex vivo megakaryocytopoiesis of serum-free expanded CD34⁺ cells. The experiment was conducted essentially in accordance with the procedures as set forth in Example 2, except that serum-free expanded CD34⁺ cells were divided into three groups and incubated in the indicated media as follows:
(1) HSA group: the SF-Mk medium as established in the above Example 1;
(2) BSA group: IMDM supplemented with a serum substitute consisting of 4.9 g/L BSA, 2.72 μg/mL insulin, and 80 μg/mL transferrin (based on the applicants' previous study, unpublished data) and the reference cytokine cocktail as previously reported in Te-Wei Chen et al. (2009), supra; and
(3) FBS group: IMDM supplemented with 10% FBS and the reference cytokine cocktail.

After 1-week induction, the Mk count of each group was calculated.

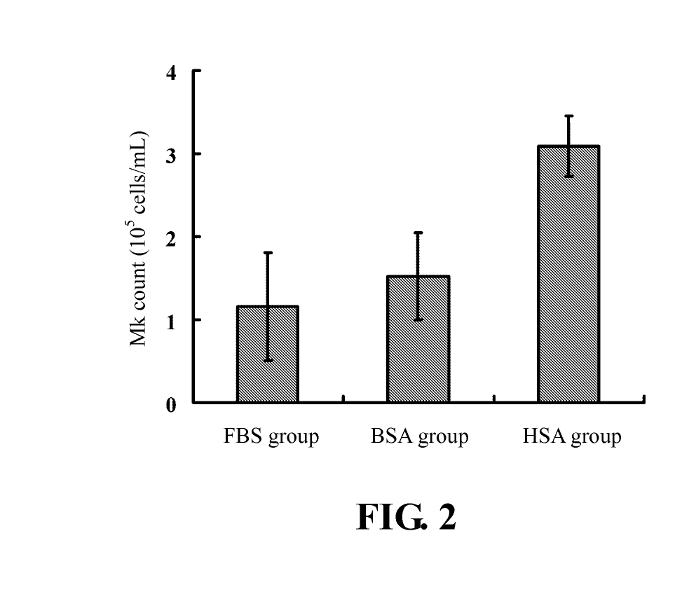

FIG. 2 is a bar diagram showing the Mk count of each group after induction for 1 week. It can be seen from FIG. 2 that the highest Mk count is observed in the HSA group (3.09±0.36×10⁵ cells/mL). It is therefore concluded that HSA is more beneficial than BSA and FBS in the formulation of a cultivating medium for the ex vivo production of megakaryocytes from human CD34⁺ cells.

### Example 4

### Comparison of the Ex Vivo Megakaryocytopoietic Potential of Freshly Isolated CD34⁺ Cells and Serum-Free Expanded CD34⁺ Cells by SF-Mk Medium

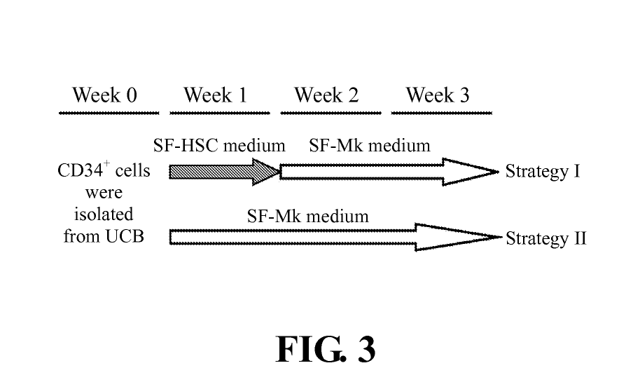

Two culture strategies as outlined in FIG. 3 were designed to compare the ex vivo megakaryocytopoietic potential of freshly isolated CD34⁺ cells and serum-free expanded CD34⁺ cells. Freshly isolated CD34⁺ cells as obtained according to the procedures set forth in Section “1. Isolation of CD34⁺ cells from human umbilical cord blood” of the General Experimental Procedures were initially seeded into each well of a 24-well plate at a concentration of 5×10⁴ cells/mL at week 0. In strategy I, freshly isolated CD34⁺ cells were incubated in the SF-HSC medium for one week and then in SF-Mk medium for two weeks. In strategy II, freshly isolated CD34⁺ cells were incubated in the SF-Mk medium for three weeks. Culture conditions were set at 37° C. and 5% CO₂ with atmospheric humidity. The medium was changed and cell density was re-adjusted to 5×10⁴ cells/mL using the SF-Mk medium at weekly intervals. The cells induced via strategies I and II were collected at weeks 0, 1, 2, and 3, and were subjected to the following analyses, respectively.

### A. Analysis of Cell Surface Antigen:

Analysis of cell surface antigen was performed based on the procedures as set forth in Section “4. Analysis of cell surface antigen” of the General Experimental Procedures, in which CD41a-FITC was used in combination with CD34-PE or CD61-PE.

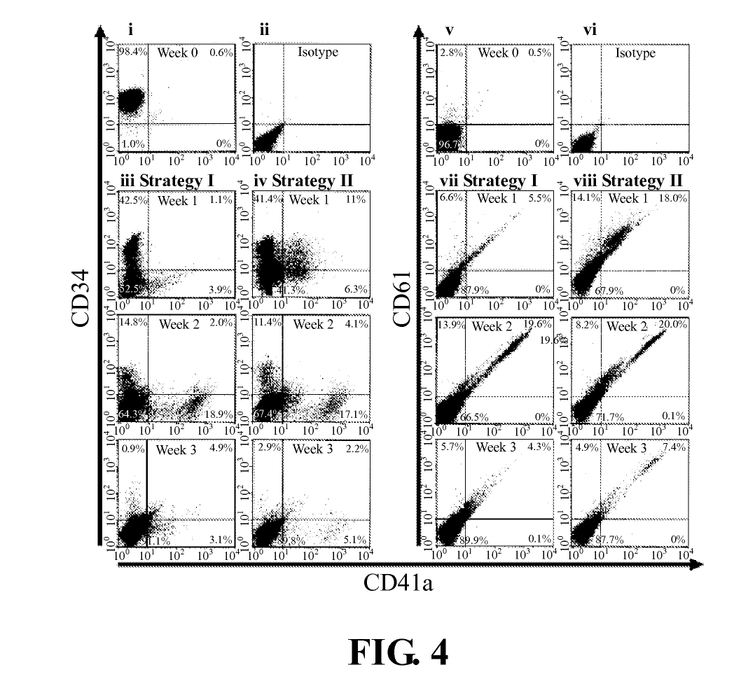

FIG. 4 shows the flow cytometry analysis of cell surface antigen expression of the cells generated from CD34⁺ cells via the culture strategies I and II of FIG. 3, respectively. It can be seen from FIG. 4 that after CD34 MultiSort MicroBeads isolation (week 0), the purity of the freshly isolated CD34⁺ cells was over 98.0% (FIG. 4i) and CD41a⁺CD61⁺ cells were almost undetectable (FIG. 4v). In strategy I, CD34 expression decreased gradually (FIG. 4iii), whereas CD41a and CD61 expression began to be detectable (FIG. 4vii). The highest percentages of CD41a⁺CD34⁺ cells and CD41a⁺CD61⁺ cells in the total cultured cells were 4.9% at week 3 and 19.6% at week 2, respectively. In strategy II, the highest percentages of CD41a⁺CD34⁺ cells and CD41a⁺CD61⁺ cells in the total cultured cells were 11.0% at week 1 (FIG. 4iv) and 20.0% at week 2 (FIG. 4viii), respectively. Decline of the co-expression of CD41a and CD61 after prolonged culture in the SF-Mk medium was observed in both strategies I and II.

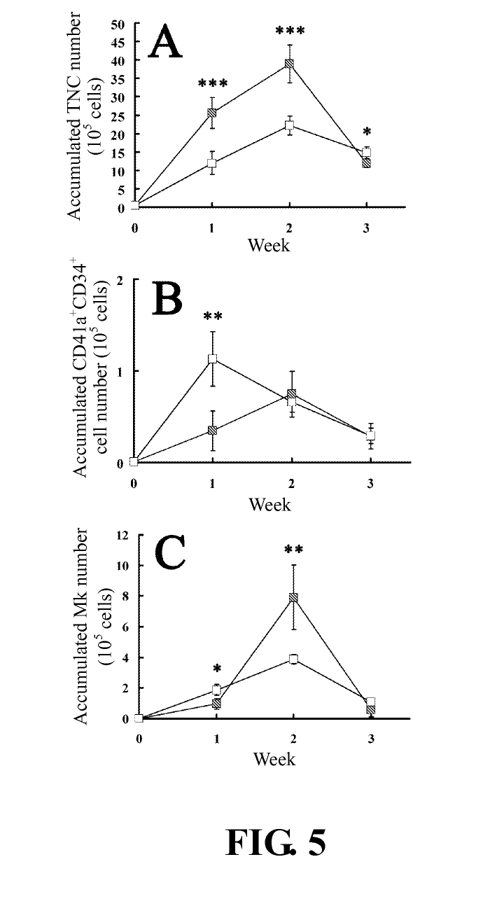

The applicants further compared the growth kinetics of cells generated via the culture strategies I and II starting from the same initial amount of freshly isolated CD34⁺ cells. The obtained results are shown in FIG. 5.

Referring to FIG. 5A, the TNC number increased more rapidly and exhibited greater expansion in strategy I than in strategy II. Referring to FIGS. 5B and 5C, subpopulations of CD41a⁺CD34⁺ cells and Mks in the freshly isolated CD34⁺ cells were almost undetectable at week 0. The maximum numbers of accumulated CD41a⁺CD34⁺ cells and Mks generated from freshly isolated CD34⁺ cells were 0.7±0.2×10⁵ (117±35-fold versus initial CD41a⁺CD34⁺ cell number at week 0) and 7.9±1.1×10⁵ (4,058±181-fold versus initial Mk number at week 0) at week 2 in strategy I, respectively, as compared to 1.1±0.3×10⁵ (at week 1, 177±40-fold versus initial CD41a⁺CD34⁺ cell number at week 0) and 3.9±0.3×10⁵ (at week 2, 1,191±368-fold versus initial Mk number at week 0) in strategy II.

### B. Analysis of DNA Content:

The cells cultured via strategies I and II were collected at weeks 1 and 2, respectively, isolated with CD41a MultiSort MicroBeads using the Miltenyi VarioMACS device, and placed into a microfuge tube. After centrifugation (700×g. 5 minutes, 4° C.), the supernatant was aspirated. The pellet was admixed with 1 mL of FACS buffer and inverted approximately three times to wash the cells. The CD41a⁺ cells were then labeled with CD41a-FITC in the dark at 4° C. for 30 minutes, followed by centrifugation (700×g, 5 minutes, 4° C.) to precipitate labeled CD41a⁺ cells. The labeled CD41a⁺ cells were perforated using a FACS™ permeabilizing solution 2 (Becton-Dickinson) and then stained with a propidium iodide (PI) staining solution (20 μg/mL PI, 0.1% Triton-X, and 0.2 mg/mL RNase in D-PBS) (Sigma) according to the procedures as previously reported (Miyazaki R et al. (2000), Br. J. Haematol., 108:602-609). DNA content was analyzed on the FACSCalibur analyzer. The experiment was repeated three times.

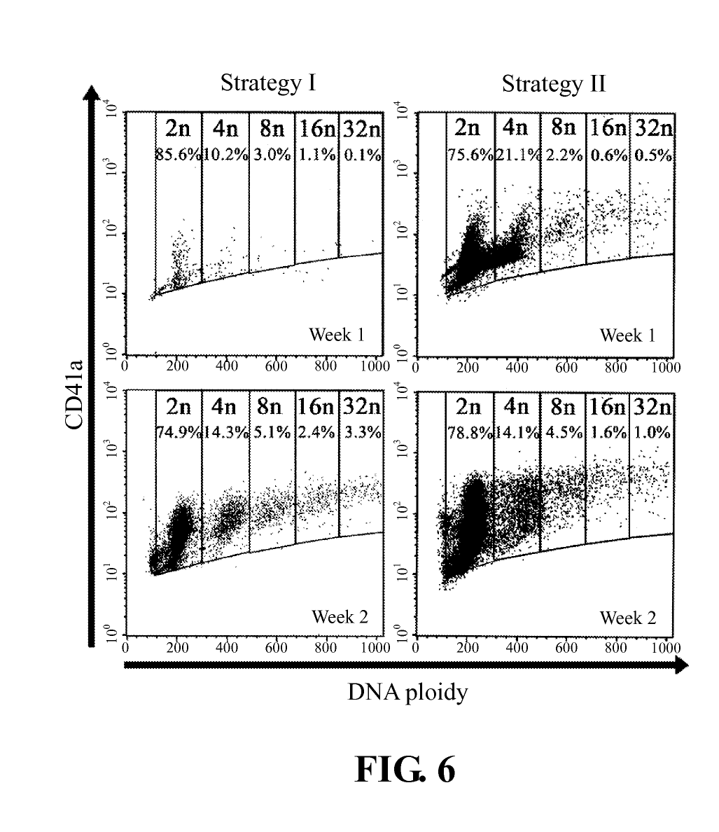

FIG. 6 shows the DNA ploidy distribution of Mks generated from CD34⁺ cells via the culture strategies I and II at weeks 1 and 2 as analyzed by flow cytometry. It can be seen from FIG. 6 that serum-free expanded CD34⁺ cells were almost diploid (2n) at week 1 in strategy I. CD41a⁺ cells with hyperploidy (>4n) were obtained when freshly isolated CD34⁺ cells or serum-free expanded CD34⁺ cells were incubated in the SF-Mk medium. CD41a⁺ cells from serum-free expanded CD34⁺ cells had a slightly higher level of hyperploidy (10.8±1.3%, at week 2 in strategy I) than those from freshly isolated CD34⁺ cells (7.1±0.9%, at week 2 in strategy II).

### C. Reverse Transcription-Polymerase Chain Reaction (RT-PCR) Analysis:

At weeks 0, 1, 2, and 3, the mRNA expression of megakaryocyte-lineage transcription factors NF-E2 and GATA-1 in the cells cultured via strategies I and II was analyzed by RT-PCR. Firstly, total RNA was extracted from the cultured cells with a Tri-Reagent (Molecular Research Center, Cincinnati, Ohio) according to the manufacturer's protocols. An equal amount of mRNA was reversed transcribed into cDNA using a First Strand cDNA Synthesis Kit (Fermentas Inc., Glen Burnie, Md.). Two gene-specific primer pairs used for reverse transcription are listed in Table 5. In addition, glyceraldehyde-3-phosphate dehydrogenase (GAPDH) mRNA expression was used as internal control. The resultant cDNA was amplified by PCR using Platinum® Taq DNA Polymerase (Invitrogen, Carlsbad, Calif.) and the amplification program was set as previously reported (Mattia G at al. (2002), Blood, 99:888-897). The PCR products were separated on a 2% agarose gel, and DNA bands were stained with ethidium bromide for visualization.

TABLE 5
Specific primer pairs used in the RT-PCR
experiment

| Target | | | |
|---|---|---|---|
| gene | Primer | Sequence (5′→3′) | |
| NF-E2 | F1 | ctactcactcatgcccaa | (SEQ ID NO: 1) |
| | R1 | ggtgctggaaaatgtca | (SEQ ID NO: 2) |
| GATA-1 | F2 | ctccctgtccccaatagtgc | (SEQ ID NO: 3) |
| | R2 | gtccttcggctgctcctgtg | (SEQ ID NO: 4) |
| GAPDH | F3 | agcctcaagatcatcagcaatg | (SEQ ID NO: 5) |
| | R3 | ttttctagacggcaggtcagg | (SEQ ID NO: 6) |

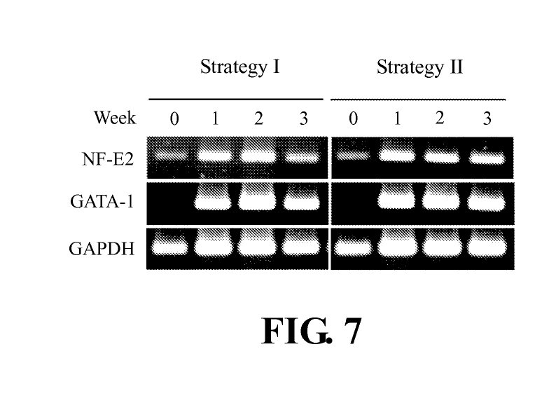

FIG. 7 shows the mRNA expression of two megakaryocyte-lineage transcription factors NF-E2 and GATA-1 in the cells cultured via strategies I and II at weeks 0, 1, 2, and 3 as determined by RT-PCR. It can be seen from FIG. 7 that freshly isolated CD34⁺ cells showed a low level of NF-E2 and GATA-1 mRNA expression at week 0. Cells generated from CD34⁺ cells via both strategies I and II began to express NF-E2 and GATA-1 clearly at week 1. NF-E2 expression (1.5-fold vs day 0) and GATA-1 expression (375.5-fold vs week 0) in strategy I reached the maximum at week 2 and decreased at week 3. In the meantime, NF-E2 expression (1.5-fold vs week 0) in strategy II was maintained at the same level throughout the 3-week Mk induction period. However, GATA-1 expression (123.4-fold vs week 0) in strategy II reached the maximum at week 1 and then slightly decreased after week 2.

### D. Analysis of Platelet Activation Ability:

In this experiment, the CD41a⁺ cells isolated after induction of CD34⁺ cells via the culture strategies I and II at week 2 were used to assess their ability to produce activated platelets using a platelet activating reagent. CD62P Expression was used as a marker for platelet activation.

Briefly, the CD34⁺ cells cultured via the culture strategies I and II were collected at week 2, respectively, and isolated with CD41a MultiSort MicroBeads using the Miltenyi VarioMACS device. CD41a MultiSort MicroBeads-isolated CD41a⁺ cells (5×10⁵ CD41a⁺ cells) were placed into a microfuge tube and subjected to centrifugation (700×g, 5 minutes, 4° C.). After removal of the supernatant, 1 mL of a platelet activating reagent (0.02 mM adenosine diphosphate, 0.19 mg/mL collagen, and 0.1 mM epinephrine) (Sigma) was added and allowed to react at room temperature for 20 minutes. The resultant mixture was washed with FACS buffer (700×g, 5 minutes, 4° C.) and then incubated with CD41a-FITC and CD62P-PE so as to label CD41a⁺CD62P⁺ cells. The expression of CD41a and CD62P was analyzed on the FACSCalibur analyzer.

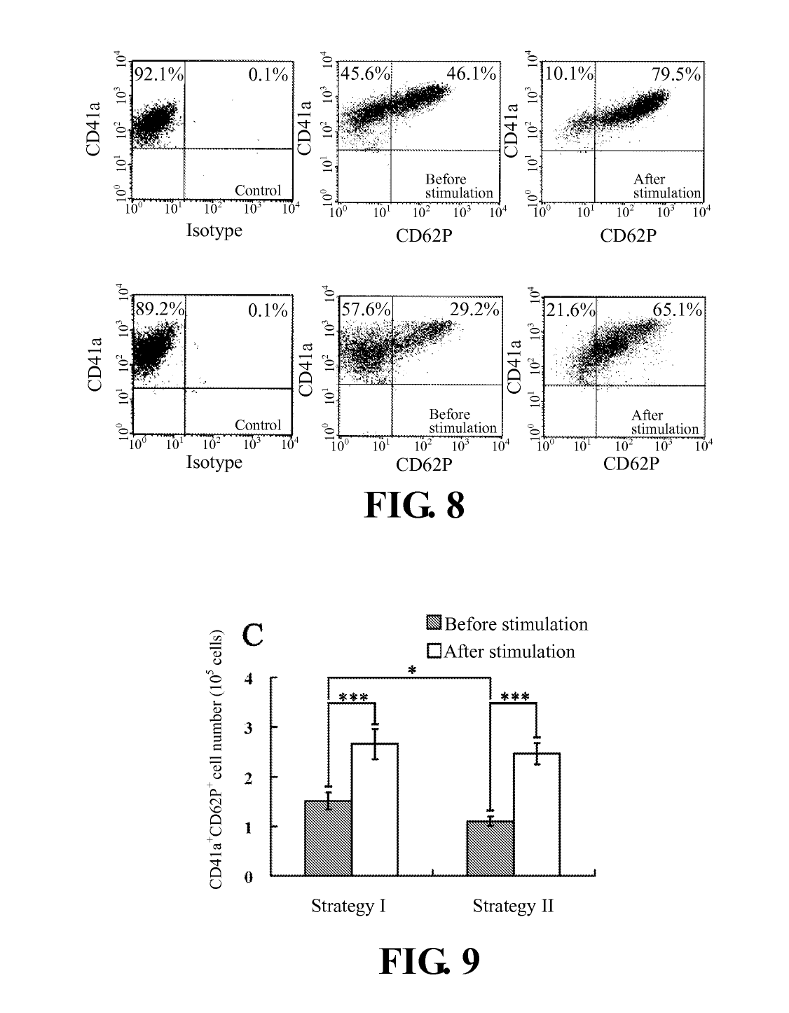

FIG. 8 shows the cell surface antigen expression of the isolated CD41a⁺ cells before and after stimulation with the platelet activating reagent, as analyzed by flow cytometry. FIG. 9 is a bar diagram showing that after stimulation with the platelet activating reagent, CD62P is significantly upregulated in the isolated CD41a⁺ cells. Referring to FIGS. 8 and 9, before stimulation with the platelet activating reagent, the cells in the strategy I and strategy II groups were measured to comprise CD41a⁺CD62P⁺ cells in a number of 1.51±0.17×10⁵ cells and 1.1±0.1×10⁵ cells, respectively. After stimulation, the CD41a⁺CD62P⁺ cells in the strategy I and strategy II groups were increased to 2.66±0.31×10⁵ and 2.46±0.21×10⁵, respectively. These results show that the culture strategies I and II could both generate Mks with the ability to become active platelets.

### Example 5

### Hematopoietic Reconstitution in X-Ray Irradiated NOD/SCID Mice by Co-Transplantation of CD34⁺ Cells and Mks

### Experimental Materials and Procedures:

### A. Experimental Animals:

NOD/SCID mice (five- to eight-weeks-old) purchased from the National Health Research Institute (Zhunan, Taiwan) were used in the following animal experiments. All the animals were housed in microisolators in laminar flow racks and were fed with autoclaved food and water. All animal experiments were performed in accordance with institutional guidelines approved by the animal ethical committee of the Food Industry Research and Development Institute (Hsinchu, Taiwan).

B. Transplantation of Human Cells into X-Ray Irradiated NOD/SCID Mice:

At day 0, the NOD/SCID mice were treated with 160 to 180 cGy total-body X-ray irradiation by using a RS 2000 X-ray Biological Irradiator (Rad Source Technologies, Inc., Alpharetta, Ga.). Two to four hours after irradiation, the mice were randomly divided into four groups (n=8 in each group) and were injected via the tail vein with:
(1) Group 1: 0.1 mL D-PBS as a negative control at day 0;
(2) Group 2: 5×10⁵ serum-free expanded CD34⁺ cells (as isolated by CD34 MultiSort MicroBeads at week 1 in strategy I) in 0.1 mL D-PBS at day 0;
(3) Group 3: 5×10⁵ serum-free generated CD61⁺ cells (as isolated by CD61 MultiSort MicroBeads at week 2 in strategy I) in 0.1 mL D-PBS at day 0: and
(4) Group 4: 5×10⁵ serum-free expanded CD34⁺ cells (as isolated by CD34 MultiSort MicroBeads at week 1 in strategy I) in 0.1 mL D-PBS at day 0, and 5×10⁵ serum-free generated CD61⁺ cells (as isolated by CD61 MultiSort MicroBeads at week 2 in strategy I) in 0.1 mL D-PBS at day 7.

### C. Detection of Human Platelet in the Peripheral Blood of Irradiated NOD/SCID Mice:

A total of 0.2 mL peripheral blood (PB) was collected with citrate-phosphate-glucose anticoagulant via a small tail incision at days 0, 9, 11 and 14, respectively. Aliquots (0.1 mL) of the thus-collected PB were used for total platelet count using a Sysmex KX-21N Hematology Analyzer (Sysmex corporation, Hamburg, Germany), which enables the measurement of the absolute number of circulating platelets. Thereafter, the total volume after collection was measured and corrected for dilution. RBCs in PB were lysed by ACK RBC lysis buffer (0.15 M NH₄Cl, 10 mM KHCO₃, 0.1 mM Na₂-EDTA, pH=7.2). The remaining cells in PB (0.1 mL) were washed with D-PBS by centrifugation at 110×g for 10 minutes and were incubated with anti-mouse FcR Blocking Reagent (Miltenyi Biotec) at 4° C. for 5 minutes. Human platelets in PB were detected by staining with CD61-PE for 30 minutes and analyzed by flow cytometry. The intensity in forward scatter (FSC), which represents the cell size, was also measured by flow cytometry. The human platelet count (human platelets/μL) was calculated by multiplying the percentage of human platelets as measured by flow cytometry by the total platelet count.

### D. Detection of Human Mk in the Bone Marrow of Irradiated NOD/SCID Mice:

14 days after transplantation, the mice were sacrificed by CO₂ euthanasia and BM was harvested from the femur by flushing with D-PBS. RBCs in BM were lysed by ACK RBC lysis buffer. The remaining cells were washed twice with D-PBS and then identified by labeling with CD45-FITC for human leukocyte and CD61-PE for human Mk. Thereafter, the labeled cells were analyzed by flow cytometry.

### Results:

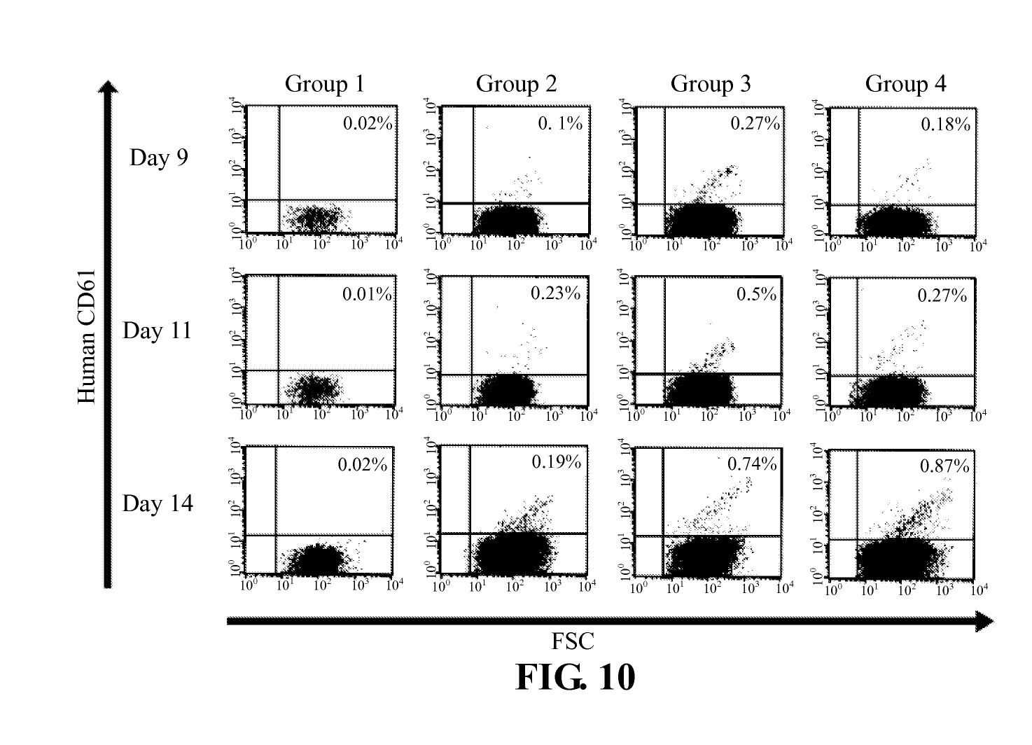

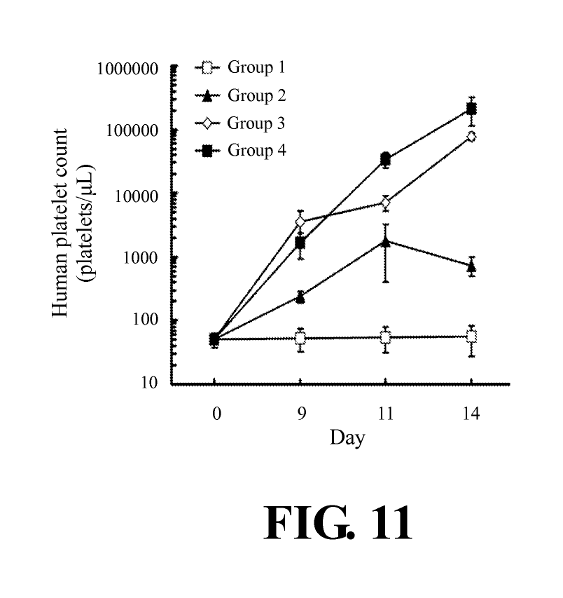

In this Example, the applicants sought to test whether the transplantation of serum-free generated Mks could rapidly boost platelet recovery. FIG. 10 shows the flow cytometry analysis of cell surface antigen CD61 expression and the cell size of the total platelets in the PB of irradiated NOD/SCID mice at days 9, 11, and 14, whereas FIG. 11 shows the growth kinetics of human platelets production in the irradiated NOD/SCID mice.

It can be seen from FIG. 10 that Group 1 did not show human platelet production at any time point. In Groups 2, 3, and 4, human platelets were detected in the PB of irradiated NOD/SCID mice at day 9 after transplantation. Human platelet percentages increased gradually until day 14, with the exception of the human platelet percentage in Group 2 (which decreased starting from the eleventh day after transplantation). At day 14 after transplantation, Group 4 showed the highest human platelet percentage (0.87±0.4%). This value was slightly higher than that of Group 3 (0.74±0.4%) and significantly higher than that of Group 2 (0.19±0.03%) (p<0.001). A similar tendency is observed in FIG. 11.

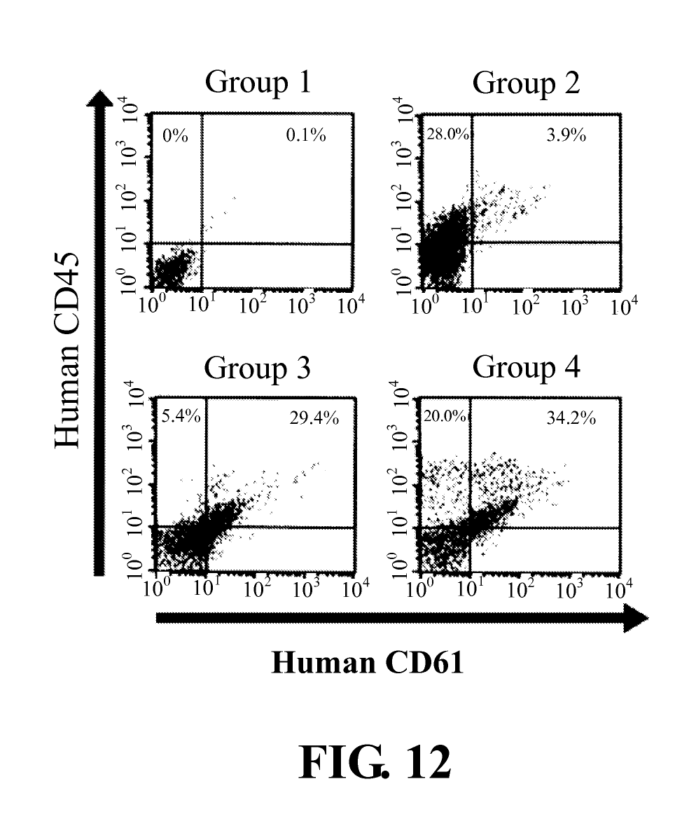

FIG. 12 shows the representative flow cytometry analysis of human Mks in the bone marrow (BM) of irradiated NOD/SCID mice at day 14 after transplantation, in which human Mks were defined as CD45⁺CD61⁺ cells. In Group 1, human cells could not be detected. In Group 2, a high percentage (28.0%) of human CD45⁺ cells but a low percentage (3.9%) of human CD45⁺CD61⁺ cells were detected in the BM of the mice transplanted with serum-free expanded CD34⁺ cells only. In Group 3, a low percentage (5.4%) of human CD45⁺ cells but a high percentage (29.4%) of human CD45⁺CD61⁺ cells were detected in the BM of the mice transplanted with serum-free generated Mks only. In Group 4, high percentages of both human CD45⁺ cells (20%) and CD45⁺CD61⁺ cell (34.2%) were detected in the BM of the mice transplanted with both serum-free expanded CD34⁺ cells at day 0 and serum-free generated Mks at day 7. These results demonstrate that platelet recovery was postponed after CD34⁺ cell transplantation. Transfusion of Mks was only able to accelerate platelet and Mk recovery in a short time, with no effects on the recovery of other mature blood constituents (Group 3). The combination of CD34⁺ cell transplantation and Mk transfusion could reconstruct BM function and rapidly recover Mk and platelet concentrations.

In conclusion, the Applicants developed the SF-Mk medium using a systematic design. In contrast to commercial media or other reports, the SF-Mk medium has a low concentration of cytokines, low induction period, and high induction efficiency. After serum-free HSC expansion and serum-free Mk induction, the increase of Mk numbers was >4.000-fold. Importantly, the identity of serum-free generated Mks was confirmed via phenotypic characteristics and functional analyses. The complete process for HSC expansion and Mk induction under serum-free conditions can provide a promising source of Mks and platelets for clinical applications and Mk therapy in the future.

All patents and literature references cited in the present specification as well as the references described therein, are hereby incorporated by reference in their entirety. In case of conflict, the present description, including definitions, will prevail.

While the invention has been described in connection with specific embodiments thereof, it will be understood that it is capable of further modifications and this application is intended to cover any variations, uses, or adaptations of the invention following, in general, the principles of the invention and including such departures from the present customary practice within the art to which the invention pertains and as may be applied to the essential features hereinbefore set forth, and as follows in the scope of the appended claims.

We claim:

1. A process for the ex vivo production of megakaryocytes from human CD34⁺ cells, comprising:
cultivating a population of human CD34⁺ cells in a cultivating medium consisting essentially of a basal medium, a serum substitute and a cytokine cocktail, wherein:
the serum substitute consists essentially of human serum albumin, insulin, and transferrin; and
the cytokine cocktail consists essentially of thrombopoietin, stem cell factor, Flt-3 ligand, interleukin-3, interleukin-6, interleukin-9, and granulocyte-macrophage colony-stimulating factor; and
harvesting a population of megakaryocytes thus formed from the cultivation of the population of human CD34⁺ cells.

2. The process according to claim 1, wherein the basal medium is selected from the group consisting of an Iscove's modified Dulbecco's medium, a Dulbecco's modified Eagle's medium, a RPMI 1640 medium, a minimum essential medium alpha medium, a basal medium Eagle medium, an F-12K nutrient mixture medium, and a Medium 199.

3. The process according to claim 2, wherein the basal medium is an Iscove's modified Dulbecco's medium.

4. The process according to claim 1, wherein based on the volume of the cultivating medium, the human serum albumin in the serum substitute is present at a concentration ranging from 4.0 to 32.0 g/L.

5. The process according to claim 1, wherein based on the volume of the cultivating medium, the insulin in the serum substitute is present at a concentration ranging from 0.9 to 7.2 μg/mL.

6. The process according to claim 1, wherein based on the volume of the cultivating medium, the transferrin in the serum substitute is present at a concentration ranging from 25.3 to 202.0 μg/mL.

7. The process according to claim 1, wherein based on the volume of the cultivating medium, the thrombopoietin in the cytokine cocktail is present at a concentration ranging from 1.8 to 13.2 ng/mL.

8. The process according to claim 1, wherein based on the volume of the cultivating medium, the stem cell factor in the cytokine cocktail is present at a concentration ranging from 7.5 to 55.0 ng/mL.

9. The process according to claim 1, wherein based on the volume of the cultivating medium, the Flt-3 ligand in the cytokine cocktail is present at a concentration ranging from 0.8 to 6.1 ng/mL.

10. The process according to claim 1, wherein based on the volume of the cultivating medium, the interleukin-3 in the cytokine cocktail is present at a concentration ranging from 1.7 to 12.7 ng/mL.

11. The process according to claim 1, wherein based on the volume of the cultivating medium, the interleukin-6 in the cytokine cocktail is present at a concentration ranging from 0.3 to 2.1 ng/mL.

12. The process according to claim 1, wherein based on the volume of the cultivating medium, the interleukin-9 in the cytokine cocktail is present at a concentration ranging from 1.0 to 7.5 ng/mL.

13. The process according to claim 1, wherein based on the volume of the cultivating medium, the granulocyte-macrophage colony-stimulating factor in the cytokine cocktail is present at a concentration ranging from 4.4 to 32.1 ng/mL.

14. The process according to claim 1, wherein the basal medium is an Iscove's modified Dulbecco's medium; and based on the volume of the cultivating medium, the serum substitute consists essentially of 8 g/L human serum albumin, 1.8 μg/mL insulin and 50.5 μg/mL transferrin, and the cytokine cocktail consists essentially of 3.0 ng/mL thrombopoietin, 12.5 ng/mL stem cell factor, 1.4 ng/mL Flt-3 ligand, 2.9 ng/mL interleukin-3, 0.5 ng/mL interleukin-6, 1.7 ng/mL interleukin-9 and 7.3 ng/mL granulocyte-macrophage colony-stimulating factor.

15. The process according to claim 1, wherein the population of human CD34⁺ cells is any one of the following:
(i) a population of human CD34⁺ cells freshly isolated from a newborn's cord blood; and
(ii) a population of human CD34⁺ cells that have been subcultured in an expansion medium after isolation from a newborn's cord blood.

16. A cultivating medium for the ex vivo production of megakaryocytes from human CD34⁺ cells, the medium consisting essentially of a basal medium, a serum substitute and a cytokine cocktail, wherein:
the serum substitute consists essentially of human serum albumin, insulin, and transferrin; and
the cytokine cocktail consists essentially of thrombopoietin, stem cell factor, Flt-3 ligand, interleukin-3, interleukin-6, interleukin-9, and granulocyte-macrophage colony-stimulating factor.

17. The cultivating medium according to claim 16, wherein the basal medium is selected from the group consisting of an Iscove's modified Dulbecco's medium, a Dulbecco's modified Eagle's medium, a RPMI 1640 medium, a minimum essential medium alpha medium, a basal medium Eagle medium, an F-12K nutrient mixture medium, and a Medium 199.

18. The cultivating medium according to claim 17, wherein the basal medium is an Iscove's modified Dulbecco's medium.

19. The cultivating medium according to claim 16, wherein based on the volume of the cultivating medium, the human serum albumin in the serum substitute is present at a concentration ranging from 4.0 to 32.0 g/L.

20. The cultivating medium according to claim 16, wherein based on the volume of the cultivating medium, the insulin in the serum substitute is present at a concentration ranging from 0.9 to 7.2 μg/mL.

21. The cultivating medium according to claim 16, wherein based on the volume of the cultivating medium, the transferrin in the serum substitute is present at a concentration ranging from 25.3 to 202.0 μg/mL.

22. The cultivating medium according to claim 16, wherein based on the volume of the cultivating medium, the thrombopoietin in the cytokine cocktail is present at a concentration ranging from 1.8 to 13.2 ng/mL.

23. The cultivating medium according to claim 16, wherein based on the volume of the cultivating medium, the stem cell factor in the cytokine cocktail is present at a concentration ranging from 7.5 to 55.0 ng/mL.

24. The cultivating medium according to claim 16, wherein based on the volume of the cultivating medium, the Flt-3 ligand in the cytokine cocktail is present at a concentration ranging from 0.8 to 6.1 ng/mL.

25. The cultivating medium according to claim 16, wherein based on the volume of the cultivating medium, the interleukin-3 in the cytokine cocktail is present at a concentration ranging from 1.7 to 12.7 ng/mL.

26. The cultivating medium according to claim 16, wherein based on the volume of the cultivating medium, the interleukin-6 in the cytokine cocktail is present at a concentration ranging from 0.3 to 2.1 ng/mL.

27. The cultivating medium according to claim 16, wherein based on the volume of the cultivating medium, the interleukin-9 in the cytokine cocktail is present at a concentration ranging from 1.0 to 7.5 ng/mL.

28. The cultivating medium according to claim 16, wherein based on the volume of the cultivating medium, the granulocyte-macrophage colony-stimulating factor in the cytokine cocktail is present at a concentration ranging from 4.4 to 32.1 ng/mL.

29. The cultivating medium according to claim 16, wherein the basal medium is an Iscove's modified Dulbecco's medium; and based on the volume of the cultivating medium, the serum substitute consists essentially of 8 g/L human serum albumin, 1.8 μg/mL insulin and 50.5 μg/mL transferrin, and the cytokine cocktail consists essentially of 3.0 ng/mL thrombopoietin, 12.5 ng/mL stem cell factor, 1.4 ng/mL Flt-3 ligand, 2.9 ng/mL interleukin-3, 0.5 ng/mL interleukin-6, 1.7 ng/mL interleukin-9 and 7.3 ng/mL granulocyte-macrophage colony-stimulating factor.

30. The cultivating medium according to claim 16, wherein the cultivating medium is used to cultivate human CD34⁺ cells freshly isolated from a newborn's cord blood or human CD34⁺ cells having been subcultured in an expansion medium after isolation from a newborn's cord blood.

31. A process for the ex vivo production of megakaryocytes from human CD34⁺ cells, comprising:
cultivating a population of human CD34⁺ cells in a cultivating medium consisting essentially of a basal medium, a serum substitute and a cytokine cocktail, wherein:
the basal medium is an Iscove's modified Dulbecco's medium;
the serum substitute consists essentially of human serum albumin, insulin, and transferrin, wherein based on the volume of the cultivating medium, the human serum albumin is present at a concentration ranging from 4.0 to 32.0 g/L, the insulin is present at a concentration ranging from 0.9 to 7.2 μg/mL, and the transferrin is present at a concentration ranging from 25.3 to 202.0 μg/mL; and
the cytokine cocktail consists essentially of thrombopoietin, stem cell factor, Flt-3 ligand, interleukin-3, interleukin-6, interleukin-9, and granulocyte-macrophage colony-stimulating factor, wherein based on the volume of the cultivating medium, the thrombopoietin is present at a concentration ranging from 1.8 to 13.2 ng/mL, the stem cell factor is present at a concentration ranging from 7.5 to 55.0 ng/mL, the Flt-3 ligand is present at a concentration ranging from 0.8 to 6.1 ng/mL, the interleukin-3 is present at a concentration ranging from 1.7 to 12.7 ng/mL, the interleukin-6 is present at a concentration ranging from 0.3 to 2.1 ng/mL, the interleukin-9 is present at a concentration ranging from 1.0 to 7.5 ng/mL, and the granulocyte-macrophage colony-stimulating factor is present at a concentration ranging from 4.4 to 32.1 ng/mL; and
harvesting a population of megakaryocytes thus formed from the cultivated population of human CD34⁺ cells.

32. A cultivating medium for the ex vivo production of megakaryocytes from human CD34⁺ cells, the medium consisting essentially of a basal medium, a serum substitute and a cytokine cocktail, wherein:
the basal medium is an Iscove's modified Dulbecco's medium;
the serum substitute consists essentially of human serum albumin, insulin, and transferrin, wherein based on the volume of the cultivating medium, the human serum albumin is present at a concentration ranging from 4.0 to 32.0 g/L, the insulin is present at a concentration ranging from 0.9 to 7.2 μg/mL, and the transferrin is present at a concentration ranging from 25.3 to 202.0 μg/mL; and
the cytokine cocktail consists essentially of thrombopoietin, stem cell factor, Flt-3 ligand, interleukin-3, interleukin-6, interleukin-9, and granulocyte-macrophage colony-stimulating factor, wherein based on the volume of the cultivating medium, the thrombopoietin is present at a concentration ranging from 1.8 to 13.2 ng/mL, the stem cell factor is present at a concentration ranging from 7.5 to 55.0 ng/mL, the Flt-3 ligand is present at a concentration ranging from 0.8 to 6.1 ng/mL, the interleukin-3 is present at a concentration ranging from 1.7 to 12.7 ng/mL, the interleukin-6 is present at a concentration ranging from 0.3 to 2.1 ng/mL, the interleukin-9 is present at a concentration ranging from 1.0 to 7.5 ng/mL, and the granulocyte-macrophage colony-stimulating factor is present at a concentration ranging from 4.4 to 32.1 ng/mL.


SEQUENCE LISTING (6 sequences)

SEQ ID NO: 1 - DNA, 18 bp, Artificial Sequence
  forward primer F1 of NF-E2 gene for RT-PCR
ctactcactcatgcccaa

SEQ ID NO: 2 - DNA, 17 bp, Artificial Sequence
  reverse primer R1 of NF-E2 gene for RT-PCR
ggtgctggaaaatgtca

SEQ ID NO: 3 - DNA, 20 bp, Artificial Sequence
  forward primer F2 of GATA-1 for RT-PCR
ctccctgtccccaatagtgc

SEQ ID NO: 4 - DNA, 20 bp, Artificial Sequence
  reverse primer R2 of GATA-1 for RT-PCR
gtccttcggctgctcctgtg

SEQ ID NO: 5 - DNA, 22 bp, Artificial Sequence
  forward primer F3 of GAPDH for RT-PCR
agcctcaagatcatcagcaatg

SEQ ID NO: 6 - DNA, 21 bp, Artificial Sequence
  reverse primer R3 of GAPDH for RT-PCR
ttttctagacggcaggtcagg